SEQUENCE LISTING (5 sequences)

SEQ ID NO: 1 - protein, 36 aa, Homo sapiens
DISGINASVVNIQKEIDRLNEVAKNLNESLIDLQEL

SEQ ID NO: 2 - protein, 36 aa, synthetic construct
IGSIDNASVVNIQKEIDRLNEVAKNLNESLIDLQEL

SEQ ID NO: 3 - RNA, 20 nt, synthetic construct
  source 1..20: mol_type = other RNA
gaccccaaaatcagcgaaat

SEQ ID NO: 4 - RNA, 24 nt, synthetic construct
  source 1..24: mol_type = other RNA
tctggttactgccagttgaatctg

SEQ ID NO: 5 - RNA, 24 nt, synthetic construct
  source 1..24: mol_type = other RNA
accccgcattacgtttggtggacc

(19) United States
(12) Patent Application Publication
(10) Pub. No.: US 2024/0108735 A1
(43) Pub. Date: Apr. 4, 2024

(54) METHODS AND COMPOSITIONS FOR TREATING COVID INFECTIONS

(71) Applicant: Decoy Therapeutics Inc., Cambridge, MA (US)

(72) Inventors: Bradley Pentelute, Revere, MA (US); Barbara L. Hibner, Winchester, MA (US)

(21) Appl. No.: 18/106,694

(22) Filed: Feb. 7, 2023

Related U.S. Application Data
(63) Continuation-in-part of application No. 17/870,158, filed on Jul. 21, 2022, now abandoned.
Application No. 17/870,158 is a continuation-in-part of application No. 17/870,174, filed on Jul. 21, 2022, now abandoned.
(60) Provisional application No. 63/307,371, filed on Feb. 7, 2022.

(51) Int. Cl. A61K 47/54; A61K 9/00; A61K 45/06; A61K 47/65; A61P 31/14

(52) U.S. Cl. CPC A61K 47/554; A61K 9/0043; A61K 45/06; A61K 47/65; A61P 31/14

(57) ABSTRACT

The invention provides a compound having the formula:

(Peptide-Linker)ₙ-B-Hydrophobic Moiety

wherein each Peptide is independently a HRC Peptide or a targeting peptide, provided that at least one peptide is a HRC Peptide, each Linker is independently a bivalent linking moiety, B is a multivalent moiety comprising cysteine, X, and optionally Y, and/or optionally Z, wherein X, Y and Z are defined herein, and n is an integer selected from 1, 2, 3 or more and methods of treating or preventing a viral infection in a subject in need thereof using the same.

### RELATED APPLICATIONS

This application is a continuation-in part of U.S. application Ser. No. 17/870,158 and U.S. application Ser. No. 17/870,174 both filed on Jul. 21, 2022. This application also claims priority to U.S. Provisional Application No. 63/307,371, filed on Feb. 7, 2022. The entire teachings of the above applications are incorporated herein by reference.

### SEQUENCE LISTING

The sequence listing submitted via EFS, in compliance with 37 CFR § 1.52(e)(5), is incorporated herein by reference. The sequence listing XML file submitted via EFS contains the file “43143001 US2 Seq List.xml”, created on Sep. 8, 2023, which is 5,505 bytes in size.

### BACKGROUND OF THE INVENTION

A variety of viruses are known to cause respiratory infections in humans, resulting in illnesses that are typically classified according to their clinical presentation, such as the common cold, influenza, bronchiolitis, croup or pneumonia. Such infections are generally self-limiting, but in certain patients, notably the elderly, infants, and those with compromised immune systems, can lead to more severe disease, including pneumonia, which can be life threatening. Most medications prescribed for these diseases provide only relief of symptoms, and there are few available drugs which modify the course of any of these diseases. In addition, new respiratory diseases caused by zoonotic viruses have recently emerged, including Severe Acute Respiratory Syndrome coronavirus (SARS-CoV), identified in 2002, Middle East Respiratory Syndrome coronavirus (MERS-CoV), identified in 2012, and, most recently, Severe Acute Respiratory Syndrome coronavirus-2 (SARS-CoV-2), which was first described in December 2019 and causes a disease referred to as COVID-19. The SARS-CoV-2 virus remains a global threat. SARS-CoV and MERS-CoV are not presently a danger to human populations, but may exist in animal reservoirs with the potential to threaten humans in the future.

In addition, the coronavirus is rapidly mutating, increasing the concern that new forms of the virus can escape existing therapies and new therapeutics will need to be developed quickly.

There remains a need in the art for methods of treating or preventing viral respiratory infections.

### SUMMARY OF THE INVENTION

The present invention is based on the discovery that by employing click chemistry to conjugate coronavirus spike proteins and other competitive ligands to lipid molecules, such as cholesterol or tocopherol new antiviral conjugates can be synthesized on demand. The method offers a programable rapid response platform for the on-demand synthesis of potent anti-viral materials. The platform includes four modules: virus fusion peptide inhibitors, membrane anchoring units (such as lipid molecules), ACE2 targeting peptides, and spike binding peptides. Covalent combinations with at least one module include a fusion peptide inhibitor conjugated to another module resulting in an effective anti-coronavirus material. Modules can be connected through a covalent framework consisting of at least one sulfur or nitrogen aryl linkage. Other modules consist of connections comprised of triazoles, amides, or sulfur-sp3 carbon bonds. These connections can be quickly made using click chemistry.

The present invention also provides compounds, compositions and methods of use thereof for treating or preventing an infection, such as compounds identified through the platform.

In one embodiment, the invention provides a compound comprising one, two, three or more HRC sequence of a viral spike peptide (also called a fusion peptide) conjugated to a membrane anchoring moiety (such as a hydrophobic moiety) via an optional linker. The hydrophobic moiety can be a membrane integrating ligand, such as a cholesterol, a sphingolipid, a glycolipid, a glycerophospholipid. The viral spike peptide is preferably a coronavirus spike protein. The peptides of the invention inhibit viral fusion.

The invention includes compositions for the delivery of compounds of the invention, such as pulmonary or nasal delivery. The invention also provides a method of treating or preventing a viral infection, including for example a SARS-CoV-2 (COVID-19) infection, in a subject in need thereof comprising administering an effective amount of a compound of the invention. The invention further provides methods of producing the compositions of the invention.

In embodiments, anti-viral compounds that are useful for treating coronavirus include a compound of the invention having a formula as shown in FIG. 1G, FIG. 1H, or FIG. 1I.

The present invention is intended to provide the efficient production of libraries of molecules. The platform and the libraries that can be produced offer the ability to interrogate variants to identify those linkers, spacers, sequences, and targeting moieties with improved antiviral activity against SARS-CoV2 and its mutations, including but not limited to the alpha, beta, gamma, delta and omicron variants.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1A through FIG. 1I depict exemplary compounds of the instant invention.

FIG. 2 depicts a schematic representation of the pseudotyped virus neutralization assay.

FIG. 3 showing the various SARS-CoV2 variants and the regions where their mutations can be found.

FIG. 4 is model of the HRC peptide from a coronavirus as described in Duquerroy et al, Virology (2005) 335:276-285.

FIG. 5 is a cross section view of an S protein trimer.

FIG. 6 illustrates atom rankings of various atoms in the amino acid side chains.

FIG. 7 is a schematic diagram of the study design of Example 2.

FIG. 8 depicts mean percentage of body weight change (y-axis), the x-axis corresponds to the period of time from −1 and −2 (base line) to Day 7.

FIG. 9 depicts percentage of body weight change (y-axis), the x-axis corresponds to the Group Treatment. An ordinary one-way ANOVA with multiple comparisons against the Control Group 1, significant differences are shown in Asterisks if present P>0.05*P≤0.05**P≤0.01***P≤0.001****P≤0.0001.

FIG. 10 depicts oral swabs viral load estimation by genome copy number (y-axis was normalized by Log₁₀) by RT-qPCR from Day 1 to Day 7 (x-axis). G1 from Day 1 to 2 had n=6 due to accidental deaths. All groups from G2 to G5 had n=8 during that period. From Day 3 to Day 7, G1 had n=3, while G2 to G5 had n=4 animals.

FIG. 11 depicts viral load estimation by genome copy number (y-axis was normalized by Log₁₀) in oropharyngeal swabs by RT-qPCR per Day, the x-axis corresponds to the Group Treatment. An ordinary one-way ANOVA with multiple comparisons against the Control Group 1, significant differences are shown in Asterisks if present ns P>0.05 * P≤0.05 ** P≤0.01 P≤0.001 **** P≤0.0001.

FIG. 12 depicts viral load estimation by genome copy number (y-axis was normalized by Log₁₀) in lungs collected on Day 2 (n=4) and Day 7 (n=4) by RT-qPCR, the x-axis corresponds to the Group Treatment. An ordinary one-way ANOVA with multiple comparisons against the Control Group 1, significant differences are shown in Asterisks if present ns P>0.05, * P≤0.05, ** P≤0.01, *** P≤0.001, or **** P≤0.0001.

FIG. 13 depicts live-SARS-CoV-2 titration assay in lungs on Study Day 2 and 7. TCID₅₀/mg of lung tissue on the y-axis was normalized using Log₁₀. Graph shows all individual animal lung titers per group (x-axis) and mean. On Day 2 Groups (2, 3, 4, and 5) had a significant reduction in viral load when compared to the Control Group 1 by TCID₅₀ (**, P<0.01, or not shown if not significant).

FIG. 14 depicts live-SARS-CoV-2 titration assay in nasal turbinates on Day 2 and 7. TCID₅₀/mg of nasal turbinate tissue on the y-axis. Graph shows all individual animal lung titers per group (x-axis) and mean. On Day 2 Groups (2, 3, 4, and 5) had a significant reduction in viral load when compared to the Control Group 1 by TCID₅₀ (**, P<0.01, or not shown if not significant).

FIG. 15 is a schematic diagram of the study design of Example 3.

FIG. 16 depicts mean percentage of body weight change (y-axis), the x-axis corresponds to the Study Days −1 and −2 (base line) to Day 7.

FIG. 17 depicts percentage of body weight change (y-axis), the x-axis corresponds to the Group Treatment. An ordinary one-way ANOVA with multiple comparisons against the Control Group 1, significant differences are shown in Asterisks if present P>0.05 * P≤0.05 ** P≤0.01 *** P≤0.001 **** P≤0.0001.

FIG. 18 depicts oral swab viral load estimation by genome copy number (y-axis was normalized by Log₁₀) by RT-qPCR from Day 1 to Day 7 (x-axis).

FIG. 19 depicts viral load estimation by genome copy number (y-axis was normalized by Log₁₀) in oropharyngeal swabs by RT-qPCR per Day, the x-axis corresponds to the Group Treatment. An ordinary one-way ANOVA with multiple comparisons against the Control Group 1, significant differences are shown in Asterisks if present ns P>0.05 * P≤0.05 ** P≤0.01 P≤0.001 ****P≤0.0001.

FIG. 20 depicts viral load estimation by genome copy number (y-axis was normalized by Log₁₀) in lungs collected on Day 2 (n=6) and Day 7 (n=6) by RT-qPCR, the x-axis corresponds to the Group Treatment. An ordinary one-way ANOVA with multiple comparisons against the Control Group 1, significant differences are shown in Asterisks if present ns P>0.05, * P≤0.05, **P≤0.01, *** P≤0.001, or **** P≤0.0001.

FIG. 21 depicts viral load estimation by TCID₅₀ (y-axis was normalized by Log₁₀) in lungs collected on Day 2 (n=6) and Day 7 (n=6), the x-axis corresponds to the Group Treatment. An ordinary one-way ANOVA with multiple comparisons against the Control Group 1, significant differences are shown in Asterisks if present n.s. are not significant differences are not shown. P>0.05, * P≤0.05, ** P≤0.01, *** P≤0.001, or **** P≤0.0001.

FIG. 22 shows efficacy of an antiviral therapeutic compound according to the invention against the WA1 strain.

FIG. 23 shows efficacy of an antiviral therapeutic compound according to the invention against Delta strain.

FIG. 24 shows efficacy of an antiviral therapeutic compound according to the invention against Omnicron strain.

FIG. 25 shows efficacy of an antiviral therapeutic compound according to the invention against P1 strain.

FIG. 26 shows efficacy of an antiviral therapeutic compound according to the invention against SARS-CoV-1 strain.

FIG. 27 shows efficacy of an antiviral therapeutic compound according to the invention against MERS-CoV strain.

FIG. 28 shows efficacy of an antiviral therapeutic compound according to the invention against OC43 strain.

### DETAILED DESCRIPTION OF THE INVENTION

Coronaviruses target human cells via the spike protein binding domain (RBD) attaching to the human angiotensin converting enzyme 2 (hACE2) receptor on host cells. The coronavirus spike (S) glycoprotein is a class I viral fusion protein on the outer envelope of the virion that plays a critical role in viral infection by recognizing host cell receptors and mediating fusion of the viral and cellular membranes. Coronavirus entry into host cells is mediated by the transmembrane spike (S) glycoprotein that forms homotrimers protruding from the viral surface. S comprises two functional subunits responsible for binding to the host cell receptor (S1 subunit) and fusion of the viral and cellular membranes (S2 subunit).

S1 serves the function of receptor-binding and contains a signal peptide (SP) at the N terminus, an N-terminal domain (NTD), and receptor-binding domain (RBD). S2 functions in membrane fusion to facilitate cell entry, and it contains a fusion peptide (FP) domain, internal fusion peptide (IFP), two heptad-repeat domains (HR1 and HR2), transmembrane domain, and a C-terminal domain.

After binding, the spike protein is activated by the host cell transmembrane protease/serine subfamily member 2 (TMPRSS2) and consequently the virus undergoes fusion with the endosomal membrane for entry into the cell. The membrane fusion domain of the spike protein in coronaviruses is highly conserved so targeting membrane fusion may result in durable long lasting therapeutics.

The invention provides a programable rapid response platform for the on-demand synthesis of potent anti-coronavirus materials. The platform preferably comprises a coronavirus fusion peptide inhibitor, a membrane anchoring moiety and a multimeric core (B) which covalently links the peptide to the membrane anchoring moiety. The platform optionally further comprises a spike binding peptide. The platform optionally further comprises a target peptide, such as an ACE2 targeting peptides or a receptor binding domain targeting peptide.

Compounds of the invention can be characterized by the following general formula:

(Peptide-Linker)ₙ-B-Hydrophobic Moiety

wherein the Linker is optional, wherein B is a multimeric core which covalently links each peptide moiety to the hydrophobic moiety and comprises cysteine, X, and optionally Y, and/or optionally Z, wherein X, Y and Z are defined herein; and wherein n is an integer selected from 1, 2, 3 or more. In a preferred embodiment, the compounds are produced by chemically conjugating each module as a discrete building block. Click chemistry is preferably used for conjugation.

Each peptide is independently a HRC peptide derived from a coronavirus spike protein and/or a targeting peptide, provided that there is at least one HRC peptide derived from a coronavirus spike protein. While preferred embodiments of the invention utilize native or wild-type peptides, non-natural peptides can be used as well. For example, amino acids found in one or more mutations (e.g., omicron mutations) can be combined with the native sequences of other viruses (e.g., the delta virus). The so-called HRC peptide or region of the coronavirus spike protein is preferred. The HRC peptides inhibit viral fusion, an important early step in the infection process. The wild type HRC peptide is a conserved region of the spike, or S, protein across coronaviruses. The conserved nature of the fusion regions (HRC/HRN) and mechanism of the class I enveloped viruses make it an ideal target to develop a pan-coronavirus inhibitor.

Preferred wild type HRC peptides comprise the sequence and binding fragments thereof:
Acₙ-DISGINASVVNIQKEIDRLNEVAKNLNESLIDLQEL (SEQ ID NO. 1), wherein n is 0 or 1.

With regard to the HRC peptide in SEQ ID NO. 1, the conventional numbering of the amino acids begins with 1150 at D. 1151I, 1154I and 1158V are in the hydrophobic interface pre-fusion and 1159V is exposed. These amino acids stabilize a helix. When the conformation change occurs (e.g., protease clipping to release FP), 1158V is exposed and 1159 V presents in the hydrophobic surface interacting with HRN trimer. 1155N, and 1176N are implicated in N-linked glycosylation conserved in coronavirus. The first 7 amino acids are implicated in HRN binding. The “N-Cap” region spans 1159V and 1171V. The 1171V is a conserved hydrophobe in coronaviruses and stabilizes the HRC hydro-core and is involved in the HRN interaction.

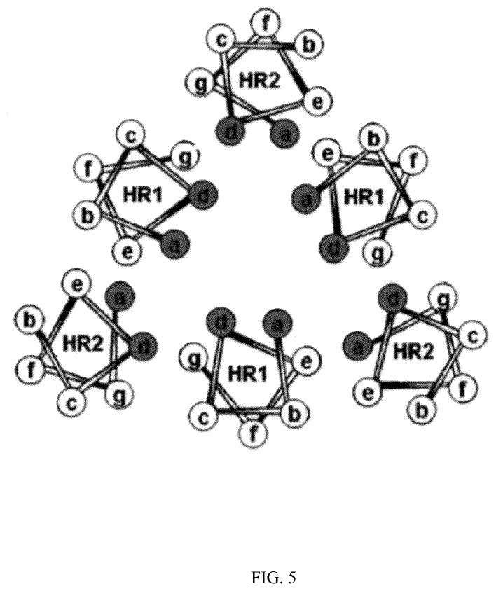

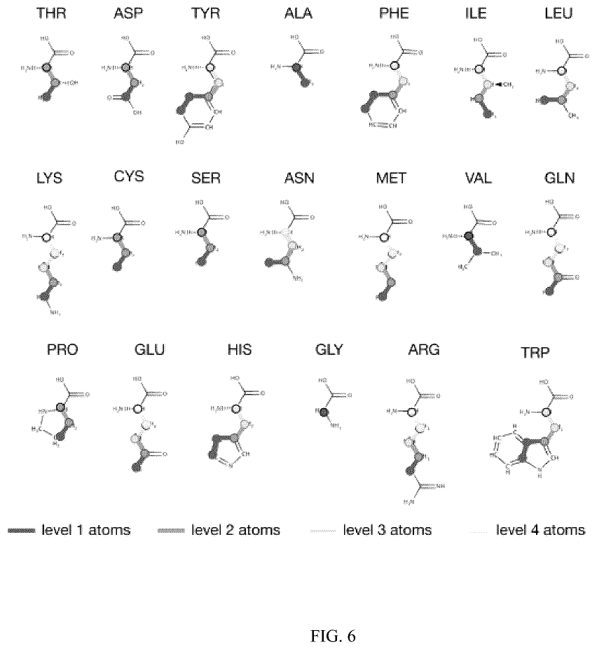

The hydrophobic core spans 1161I and 1175L and is helical pre- and post-fusion. The isoleucines, leucines and alanine are important in folding and stability of a coiled coil. The C-Cap region spans 1176N and 1185L. 1179L, 1182L and 1185L are in the hydrophobic interface pre-fusion and 1180I is exposed. These amino acids stabilize the helix. 1185Y may be implicated in hydrophobic packing between three polypeptide chains in a trimeric coiled coil. When confirmation change occurs (protease clipping to release FP), 1179L is exposed and 1180I is in the hydrophobic surface interacting with a HRN trimer. 1164E and 1184E form a salt bridge between HRC and HRN. Further, 1159V, 1160N, 1171V and 1180I have been shown to interact with HRN in crystal structures. FIG. 5 illustrates a cross section view of an S protein trimer.

In certain embodiments, the peptides are selected from variants of a wild type HRC peptides comprising the sequence and binding fragments thereof:
Acₙ-DISGINASVVNIQKEIDRLNEVAKNLNESLIDLQEL (SEQ ID NO. 1), wherein n is 0 or 1.

In the context of protein variants, the term “variant” is defined as a peptide which has at least one amino acid deleted, added, or substituted in comparison with a wild type sequence, such as SEQ ID NO. 1 or other native sequence described herein. Variants preferably bind the cognate ligand of the wild type sequence. For example, a peptide wherein 1, 2, 3, 4 or 5 amino acids of SEQ ID NO. 1 are substituted can be used. Such substituted amino acids can preferably be selected from one or more corresponding amino acids identified in a different coronavirus strain via a sequence alignment, such as shown above. For example, one or both underlined isoleucines can be substituted with leucine and/or methionine, as described in the alignment provided above. The underlined alanine can be substituted by valine, leucine or isoleucine. One or both underlined leucines can be independently substituted by isoleucine, tyrosine, alanine or valine. Other conservative or nonconservative substitutions, (lysine and glutamine or aspartic acid and glutamic acid) can be selected as well, for example, as shown in the above sequence alignment. In embodiments, amino acids that are conserved amongst 2, 3, 4, 5 or more coronavirus (e.g., coronavirus isolated from bats or SARS-CoV2 mutants or variants) remain conserved in the non-natural HRC peptide.

For example, the wild type HRC sequence can include 1, 2, 3, 4, 5, 6, 7, 8, 9, 10 or more additional amino acids native to the S protein at the N- and/or C termini. For example, glycine can be added to the N-terminus. Additionally, the wild type HRC peptide fragment can delete 1, 2, 3, 4, 5, 6, 7, 8, 9, or 10 amino acids at the N and/or C termini and inhibit infection. Typically, not more than ten total amino acids are deleted in total. For example, the 10 amino acids at the C terminus can be deleted and be expected to retain inhibitory activity.

In certain embodiments, modifications to wild type sequences are desirable. For example, using one or more D amino acids can improve pharmacokinetics and the half life of the peptide. Thus, in certain embodiments, the invention includes peptides characterized by 1, 2, 3, 4, 5, 6, 7, 8, 9, 10 or more D-amino acids. The D-amino acids can preferably be a corresponding L-amino acid of the wild type sequence. In certain embodiments, the D-amino acid is an amino acid located at or near (e.g., within 1, 2, or 3 amino acids) a protease degradation site. In certain embodiments, the D-amino acid is a hydrophobic amino acid participating in binding with the HRN peptide and preferably at a higher affinity than the corresponding wild type sequence. Alternatively or additionally, the D-amino acid is a hydrophilic amino acid, such as lysine, aspartic acid, glutamic acid or arginine. Alternatively or additionally, the D-amino acid can be selected from the 7 amino acids at the N-terminus of SEQ ID NO:1. Peptides that have been improved by incorporating D-amino acids are described in U.S. Ser. No. 63/140,387, filed on Jan. 22, 2021, which is incorporated by reference in its entirety.

However, swapping one or more D-amino acids for the corresponding L-amino acid can change the topology of the peptide and impact function. Therefore, a preferred non-natural HRC peptide is a Retro-Inversion HRC peptide, or “RI HRC peptide”. Retro-Inversion HRC peptides are preferably characterized by a binding affinity of at least about 50% of the wild-type HRC peptide with its cognate ligand in a standard binding assay and decreased susceptibility to mammalian protease degradation. Retroinversion is defined as reversing a D-peptide sequence of a helical peptide or “flipping” the termini thereby restoring the presentation of the side chains to the binding ligand or target. See Kim et al, Method to generate highly stable D-amino acid analogs of bioactive helical peptides using a mirror image of the entire PBS, PNAS, Feb. 13, 2018, 115 (7) 1505-1510, which is incorporated herein by reference in its entirety. Therefore, a non-natural peptide of the invention can include a peptide having the sequence of SEQ ID NO.:1 wherein amino acids are D-amino acids, such as the amino acids within a region, flipping the N-terminus for a C terminus. For example, the N termini can be subjected to retroinversion as shown in SEQ ID NO. 2 where each D amino acid is preceded by a “d”:
dIdGdSdIdD NASVVNIQKEIDRLNEVAKNLNESLIDLQEL (SEQ ID NO. 2)

This example offers a single RI region of 5 amino acids. However, as few as two amino acids can be selected (e.g., the 2 N-terminal amino acids). For example, the RI region can span the hydrophobic core, 1160N to 1176N, or the C-cap region or a portion thereof. Alternatively, the entire peptide can be an RI peptide. Additionally, two, three or more RI regions can be included. For example, both the N-terminus and C-Cap region can be RI regions, retaining the hydrophobic core with L-amino acids.

For example, in using mirror-image phage display to screen for HRC variants, a first D-peptide can be synthesized from a HRN coronavirus peptide, or first L-peptide. The first L-peptide can be a naturally occurring L-peptide or can be a chimera of a peptide. The methods can further comprise screening for a HRC peptide, or second L-peptide, that specifically binds to the first D-peptide; then, a second D-peptide that is the mirror image of the second L-peptide can be synthesized. In one aspect of the D-peptide screening methods described herein, an N-trimer target can first be synthesized with D-amino acids, creating the mirror image of the natural L-N-trimer target. The D-N-trimer target can be used in standard peptide-based screens such as phage display, ribosome display, and/or CIS display to identify L-peptides that bind to the D-N-trimer. The identified L-peptides can then be synthesized with D-amino acids. By the law of symmetry, the resulting D-peptides bind the natural L-N-trimer and will thus target the N-trimer region of the coronavirus HRN intermediate, thereby inhibiting infection. This screening method is also described in Schumacher, et al., Identification of D-peptide ligands through mirror-image phage display, Science, 1996 Mar. 29; 271(5257):1854-7, which is hereby incorporated in its entirety by this reference.

The hotspot residues of the HRC peptide can be identified by crystal structure or NMR solution structure of the HRC peptide. For example, 1, 2, 3, 4, 5, 6, 7, 8, 9, 10 or more amino acids selected from 1150D, 1151I, 1154I, 1155N, 1158V, 1159V, 1161I, 1164E, 1171V, 1175L, 1176L, 1179L, 1180I, 1182L, 1184E and/or 1185Y, such as one or more amino acids selected from 1159V, 1160N, 1163E, 1171V, 1180I, 1184E and/or 1185L of SEQ ID NO. 1 can be designated hotspot residues.

In embodiments, the Peptide comprises a cell targeting peptide. In embodiments, the targeting peptide is selected from but not limited to an ACE2 targeting peptide or a Receptor binding domain targeting peptide. In embodiments, the targeting peptide is an ACE2 targeting peptide. In embodiments, the targeting peptide is a Receptor binding domain targeting peptide.

In embodiments where the compound comprises only one Peptide, the peptide is HRC peptide derived from a coronavirus spike protein. In embodiments where the compound comprises 2 or more Peptides, each peptide can be a HRC peptide derived from a coronavirus spike protein. In embodiments where the compound comprises 2 or more Peptides, the Peptide can be selected from a HRC peptide derived from a coronavirus spike protein and/or a targeting peptide, provided that there is at least one HRC peptide derived from a coronavirus spike protein. Preferably, only one of the Peptides is a targeting peptide and the other peptide(s) are a HRC peptide derived from a coronavirus spike protein.

In certain embodiments, the optional “Linker” is defined as a bivalent moiety or group that covalently binds to a Peptide (preferably at the C or N terminus thereof) and to B. In embodiments, the Linker can have 1, 2, 3, 4, 5 or more subunits or segments. In embodiments, the Linker comprises a subunit with one or more amino acids. The amino acids may be naturally occurring or synthetic. Thus, the Linker may comprise (Gly)ₙ₊₁, (GlySerGly)ₙ or (Gly-Pro)ₙ where n is 1 or greater, for example, 1 to 12, 1 to 6 or 1 to 4. GlySerGly is one example of a sequence of amino acids which may form the Linker or part of the Linker.

In certain embodiments, the Linker may comprise a non-amino acid subunit. In some embodiments, examples of the non-amino acid subunit of the linker are —(OCH₂CH₂)ₘ— where m is from 1 to 15, for example 2 to 10, 2 to 6 or 4. Introduction of a (poly)ethyleneglycol group assists solublity in aqueous media. In some embodiments, examples of the non-amino acid portion of the linker are —CH₂C(O)— and —CH₂C(O)NHCH₂CH₂(OCH₂CH₂)₄C(O)—.

For example, it can be advantageous to use a Linker with 3 subunits. A first optional subunit which comprises a flexible peptide, such as-(G)ₘ- or -(GS)ₘG-, where m is an integer of 1, 2, 3, 4, 5 or more, such as 2. A second subunit can be a residue of a chemical reaction (such as a click chemistry reaction), such as a peptide bond, ester, ether, or thioether involving the N-terminus, C-terminus or side chain of the Peptide or first subunit. The residue can be non-cleavable, such as that formed with carbodiimide or sulfhydryl maleimide. A third optional subunit can be a hydrophilic spacer, such as polyethyleneglycol, polyethyleneamine, polyacetal polymer, poly(1-hydroxymethylethylene hydroxymethyl-formal) (PHF) or a carbohydrate. The hydrophilic spacers can generally be polymeric and comprise 1, 2, 3, 4, 5, 6, 7, 8, 9, 10 or more monomers. Polyethyleneglycol with 4 monomers (PEG4) is satisfactory. The length of the hydrophilic spacer can correspond to the span the S protein gap to facilitate the orientation of the peptide to bind the HRN domain. PEG can also reduce aggregation and improve solubility. Another subunit can be the residue of a chemical reaction (such as a click chemistry reaction), between B and the Linker or subunit thereof.

B can be a multimeric core which provides a framework that covalently links the one or more Peptide-Linker) moieties to the hydrophobic moiety. B can comprise one or more cysteine, one or more X, and optionally Y, and/or optionally Z, wherein X, Y and Z are defined herein. In embodiments, the one or more cysteine binds the one or more Peptide-Linker moieties to the other components of B.

There is a need to be able to covalently link peptides to B using finely tunable and simple chemical reactions that are nontoxic to cells, that can occur at a rapid rate under biological conditions. The invention addresses this need. In broad aspect, this disclosure provides click-chemistry compatible functional groups, also referred to herein as “click chemistry handles”, that react via click chemistry.

As utilized herein, it should be understood that “click-chemistry compatible” structures, functional groups, monomers, oligomers, etc., refer to compounds, materials, etc. that are structurally characterized by including one or more chemical moieties suitable for participation in a click-chemistry reaction.

Click chemistry handles are chemical moieties that provide a reactive group that can partake in a click chemistry reaction. Click chemistry reactions and suitable chemical groups for click chemistry reactions are well known to those of skill in the art, and include, but are not limited to terminal alkynes, azides, strained alkynes, dienes, dieneophiles, alkoxyamines, carbonyls, phosphines, hydrazides, thiols, and alkenes. For example, in some embodiments, an azide and an alkyne are used in a click chemistry reaction.

Some aspects of this invention provide modified Peptides, for example, proteins comprising a C-terminal or an N-terminal click chemistry handle. Such Peptides can be then be covalently conjugated to B comprising a moiety that can react with the click chemistry handle of the protein.

In embodiments where copper-catalyzed azide-alkyne cycloaddition (CuAAC) is the click-chemistry employed for functionalizing materials as disclosed herein, the “click-chemistry compatible” compounds include a terminal alkyne and/or terminal azide functional group.

An exemplary click-chemistry reaction is CuAAC, although skilled artisans will appreciate that other click-chemistry compatible reactions that would be appreciated as equivalent to CuAAC may be employed without departing from the scope of the inventive concepts described herein. For instance, in various embodiments click-chemistry compatible reactions may include CuAAC, strain-promoted azide-alkyne cycloaddition (SPAAC), strain-promoted alkyne-nitrone cycloaddition (SPANC), strained alkene reactions such as alkene-azide cycloaddition, etc. Click-chemistry compatible reactions may also be considered to include alkene-tetrazine inverse-demand Diers-Alder reactions, alkene-tetrazole photoclick reactions, Michael additions of thiols, nucleophilic substitution of thiols with amines, and certain Diels-Alder reactions, etc. such as disclosed by Becer, et al. “Click chemistry beyond metal-catalyzed cycloaddition.” Angew. Chem. Int. Ed. 2009, 48: p. 4900-4908, and equivalents thereof as would be understood by a person having ordinary skill in the art upon reading the present disclosures.

Accordingly, click-chemistry compatible groups, compounds, etc. should be understood to include one or more suitable chemical moieties conveying capability to participate in any combination of the foregoing exemplary click chemistries, in various embodiments.

B is a multimeric core which provides a framework that covalently links the one or more Peptide-Linker) moieties to the hydrophobic moiety. B comprises one or more cysteine residue, one or more X, and optionally Y, and/or optionally Z, wherein X, Y and Z are defined herein.

The one or more cysteine residue, one or more X, optional Y, and optional Z can be in any order, wherein the component of B listed first is bound to the Peptide-Linker and the component listed last is bound to the Hydrophobic Moiety. For example, wherein B comprises, in order, one or more cysteine residue, one or more X, and Z, the Peptide-Linker is bound to the one or more cysteine and Z is bound to the Hydrophobic Moiety. In embodiments, B comprises, in order, one or more cysteine and one or more X. In embodiments, B comprises cysteine and X.

In embodiments, B comprises one or more cysteine, one or more X and Z. In embodiments, B comprises Z, one or more cysteine, and one or more X.

In embodiments, B comprises Y, one or more cysteine, and one or more X. In embodiments, B comprises Y, one or more cysteine, one or more X, and Z.

In embodiments, the one or more cysteine binds the one or more Peptide-Linker moieties to the other components of B. In some embodiments, the one or more cysteine is attached to the one or more X via a thioether bond. In embodiments, the one or more cysteines have the following structure:

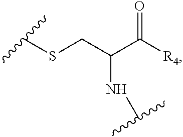

wherein R₄ is OH or NH₂.

In embodiments, X comprises one or more sulfur aryl linkage, nitrogen aryl linkage or other linkages such as triazoles, amides, sulfur-sp3 carbon bonds, or a hydrophilic linker. In embodiments, hydrophilic linker is selected from such as polyethyleneglycol (PEG), polyethyleneimine, polyacetal polymer, poly(1-hydroxymethylethylene hydroxymethyl-formal) (PHF) or a carbohydrate. In embodiments, the hydrophilic linker can be polymeric and comprise 1, 2, 3, 4, 5, 6, 7, 8, 9, 10 or more monomers. In embodiments, the hydrophilic linker is polyethyleneglycol (PEG). In embodiments, the hydrophilic linker is polyethyleneglycol with 4 monomers (PEG4). In one embodiment X comprises one or more sulfur aryl linkage. In one embodiment X comprises one or more nitrogen aryl linkage. In one embodiment, X comprises one or more sulfur-sp3 carbon bonds.

In embodiments, the one or more X is attached via a thioether bond directly with the hydrophobic moiety. In embodiments, the one or more X is attached to Z, when present, which is then attached to the hydrophobic moiety.

In certain embodiments, B further comprises Y. In embodiments, Y, when present, comes between the one or more Peptide-Linker moieties and the one or more cysteine and binds the one or more Peptide-Linker moieties to the one or more cysteine, which then binds to the other components of B.

In embodiments, Y comprises one or more amino acids. The amino acids may be naturally occurring or synthetic. Y may comprise 1 or more amino acids, for example, 1 to 12, 1 to 6 or 1 to 4. The one or more amino acids can be added to the linker in stepwise fashion. For example, a first amino acid is added to the cysteine of B and then, prior to the addition of a second amino acid, a Peptide-Linker is attached to the first amino acid. After attachment of the Peptide-Linker to the first amino acid, a subsequent amino acid is attached to the previous amino acid and allows for the attachment of a further Peptide-Linker and so on. In some embodiments, the amino acid of the linker is one or more lysines.

In embodiments, Z, when present, comes between X and the hydrophobic moiety and binds B to the hydrophobic moiety.

In embodiments, Z, when present, comprises a moiety having a structure according to formula (I):

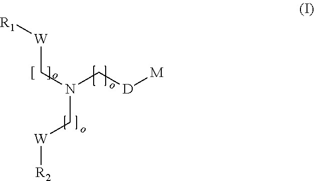

wherein each of R1 and R2 is independently selected from the group consisting of:
(i) R₃;
(ii) a structure according to formula (II):

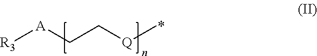

and
(iii) a structure according to formula (III):

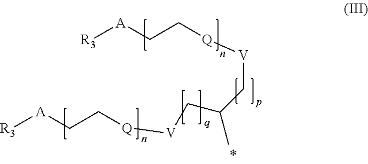

wherein

W is in each instance independently selected from —H—C(O)—O—, —O—C(O)— H—, —C(O)—O—, —O— C(O)—, —(CH₂)ₘ—, —H—C(O)—, —C(O)—H—, —H—, and —C(X)— most preferably W is —C(O)— H—;

V is in each instance independently selected from —(CH₂)ₘ—, —(CH₂)ₘ—C(X)—H—, —H—C(X)—(CH₂)ₘ—, —(CH₂)ₘ—H—C(O)—O—, —O—C(O)—H—(CH₂)ₘ—, —(CH₂)ₘ—C(O)—O—, —O—C(O)—(CH₂)ₘ—, —H—C(X)—, —C(X)—H—, —H—C(O)—O—, —O—C(O)— H—, —C(O)—O—, and —O—C(O)—; most preferably

V is —CH₂CH₂—C(O)—H—;

D is in each instance either O, S, or H;

A is in each instance independently selected from —C(O)CH₂—, —CH₂C(O)—, —HCH₂—, —CH₂H—, —HC(O)—, —C(O)H—, —H—, —CH₂—, —CH₂C(O) H— and —HC(O)CH₂—; most preferably Y is —HCH₂—;

Q is in each instance independently selected from —CH₂—, —H—, —O—, —CH₂O—, —HCH₂— and —OCH₂—; most preferably Z is —O—;

R3 is in each case independently selected from any of said polypeptides, which may be the same or different;

m is in each instance independently selected from an integer of between 0 and 5, i.e., 0, 1, 2, 3, 4, or 5; preferably between 0 and 3, preferably m is the same in each instance;

n is in each instance independently selected from an integer of between 0 and 40, i.e., 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40; preferably between 3 and 10, preferably n is the same in each instance;

o is in each case independently selected from an integer of between 0 and 5, i.e., 0, 1, 2, 3, 4, or 5; preferably 2, preferably o is the same in each instance;

p is in each instance independently selected from an integer of between 0 and 5, i.e., 0, 1, 2, 3, 4, or 5; preferably between 0 and 3, preferably p is the same in each instance;

q is in each instance independently selected from an integer of between 0 and 5, i.e., 0, 1, 2, 3, 4, or 5; preferably between 0 and 3; preferably q is the same in each instance and/or preferably q<p;

M is said Hydrophobic Moiety; and

wherein * marks, where the structures (II-III) are linked to structure (I).

In embodiments, Z, when present, does not comprises a moiety having a structure according to formula (I):

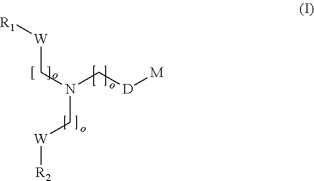

In embodiments, Z, when present comprises a moiety having a structure according to formula (IV):

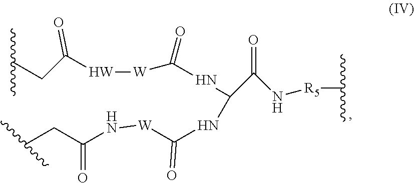

wherein R₅ is selected from hydrophilic linker is selected from such as polyethyleneglycol (PEG), polyethyleneimine, polyacetal polymer, poly(1-hydroxymethylethylene hydroxymethyl-formal) (PHF) or a carbohydrate; and
W is in each instance independently selected from direct bond, hydrophilic linker is selected from such as polyethyleneglycol (PEG), polyethyleneimine, polyacetal polymer, poly(1-hydroxymethylethylene hydroxymethyl-formal) (PHF) or a carbohydrate.

In embodiments, the hydrophilic linker of R₅ can be polymeric and comprise 1, 2, 3, 4, 5, 6, 7, 8, 9, 10 or more monomers. In embodiments, the hydrophilic linker of R₅ is polyethyleneglycol (PEG). In embodiments, the hydrophilic linker of R₅ is polyethyleneglycol with 4 monomers (PEG4).

In embodiments, the hydrophilic linker of W can be at each instance independently polymeric and comprise 1, 2, 3, 4, 5, 6, 7, 8, 9, 10 or more monomers. In embodiments, the hydrophilic linker is polyethyleneglycol (PEG). In embodiments, the hydrophilic linker of W can be at each instance independently polyethyleneglycol with 4 monomers (PEG4).

The Hydrophobic Moiety can be a membrane integrating lipid, such as cholesterol, a sphingolipid, sphingomyelin, a glycolipid, glycerophospholipid (such as phosphatidylcholine, phosphatidylethanolamine and phosphatidylserine), ergosterol, 7-dihydrocholosterol and stigmasterol. Preferably cholesterol. Typically, B is linked directly or indirectly to a cholesterol hydroxyl group, such as 3-OH. The hydrophobic moiety facilitates insertion of the compound of the invention into a cell membrane and can inhibit viral entry.

B may be connected to any convenient position on the Hydrophobic Moiety. In some embodiments, connection may be via a hydroxy group of the Hydrophobic Moiety. For example, when the Hydrophobic Moiety is cholesterol, B may be connected to the cholesterol by a group —C(O)— or —C₁₋₄alkylene C(O)—, such as —CH₂C(O)—.

Phospholipids include such lipids as egg phosphatidylcholine (EPC), egg phosphatidylglycerol (EPG), egg phosphatidylinositol (EPI), egg phosphatidylserine (EPS), phosphatidylethanolamine (EPE), and phosphatidic acid (EPA); the soya counterparts, soy phosphatidylcholine (SPC); SPG, SPS, SPI, SPE, and SPA; the hydrogenated egg and soya counterparts (e.g., HEPC, HSPC), other phospholipids made up of ester linkages of fatty acids in the 2 and 3 of glycerol positions containing chains of 12 to 26 carbon atoms and different head groups in the I position of glycerol that include choline, glycerol, inositol, serine, ethanolamine, as well as the corresponding phosphatidic acids. The chains on these fatty acids can be saturated or unsaturated, and the phospholipid may be made up of fatty acids of different chain lengths and different degrees of unsaturation. In particular, the compositions of the formulations can include dipalmitoylphosphatidylcholine (DPPC), a major constituent of naturally-occurring lung surfactant. Other examples include dimyristoylphosphatidycholine (DMPC) and dimyristoylphosphatidylglycerol (DMPG) dipalmitoylphosphatideholine (DPPQ) and dipalmitoylphosphatidylglycerol (DPPG) distearoylphosphatidylcholine (DSPQ) and distearoylphosphatidylglycerol (DSPG), dioleylphosphatidyl-ethanolarnine (DOPE) and mixed phospholipids like palmitoylstearoylphosphatidyl-choline (PSPC) and palmitoylstearolphosphatidylglycerol (PSPG), and single acylated phospholipids like mono-oleoyl-phosphatidylethanolamine (MOPE).

Cholesterols can include, cholesterol, esters of cholesterol including cholesterol hemi-succinate, salts of cholesterol including cholesterol hydrogen sulfate and cholesterol sulfate, ergosterol, esters of ergosterol including ergosterol hemi-succinate, salts of ergosterol including ergosterol hydrogen sulfate and ergosterol sulfate, lanosterol, esters of lanosterol including lanosterol hemi-succinate, salts of lanosterol including lanosterol hydrogen sulfate and lanosterol sulfate. The tocopherols can include tocopherols, esters of tocopherols including tocopherol hemi-succinates, salts of tocopherols including tocopherol hydrogen sulfates and tocopherol sulfates.

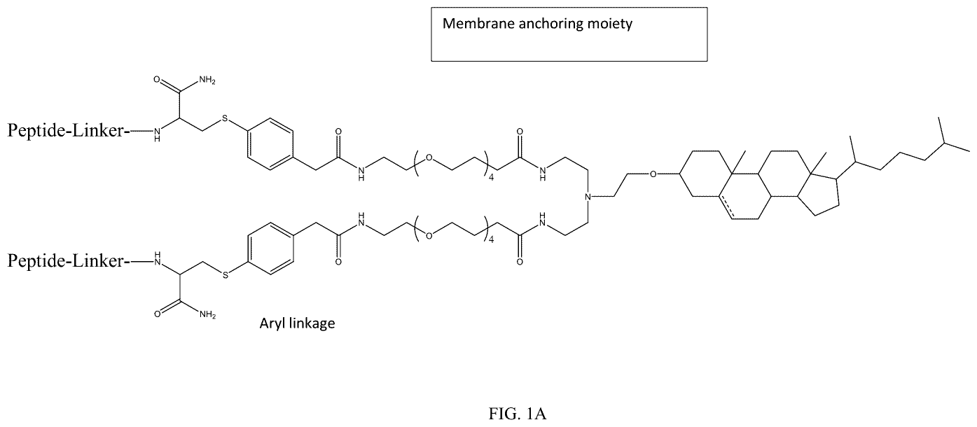

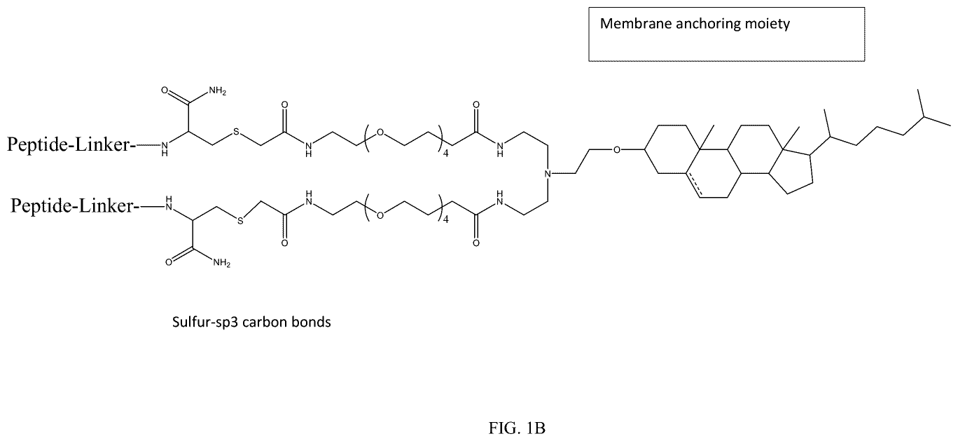

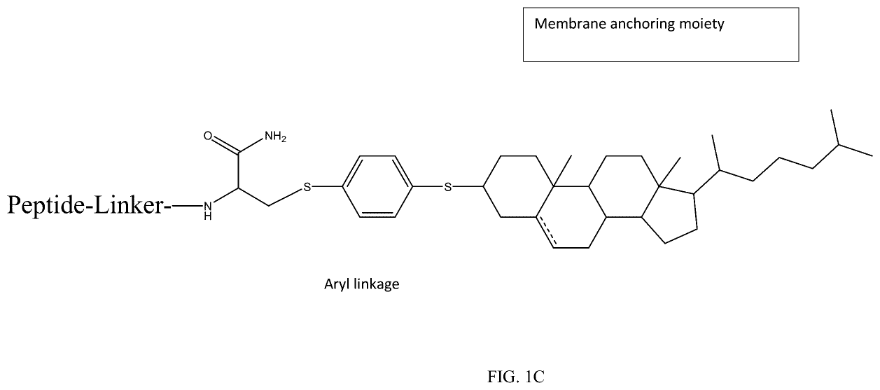

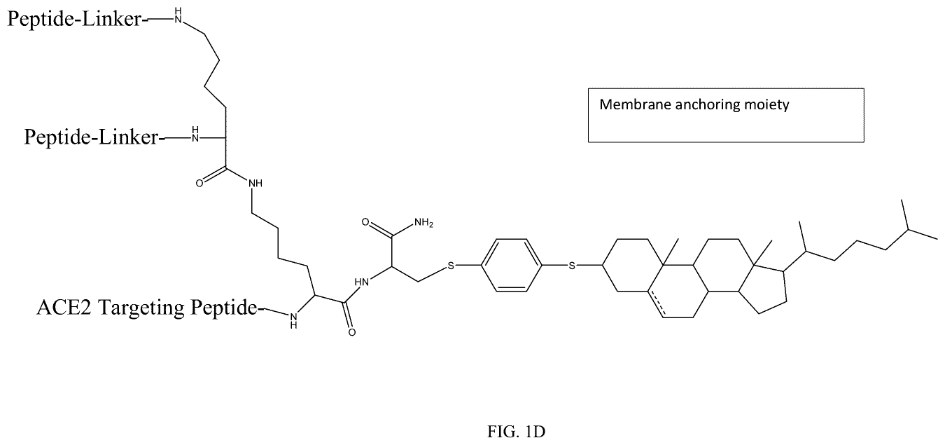

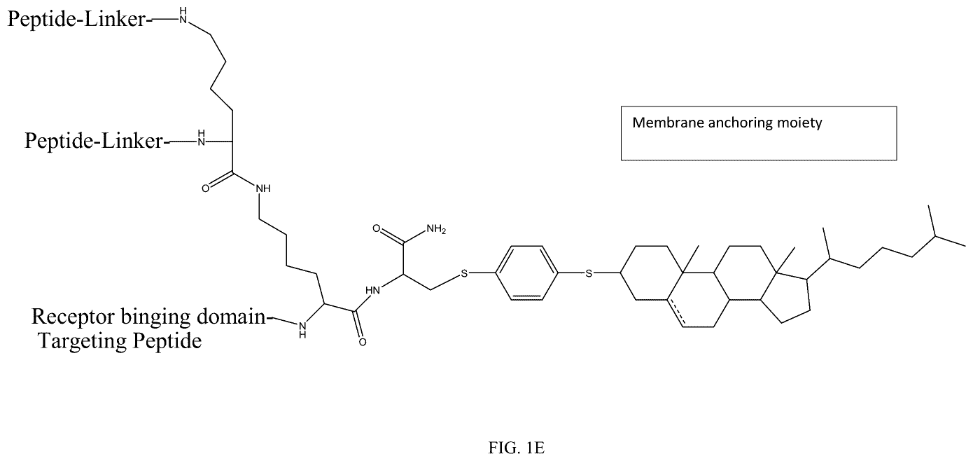

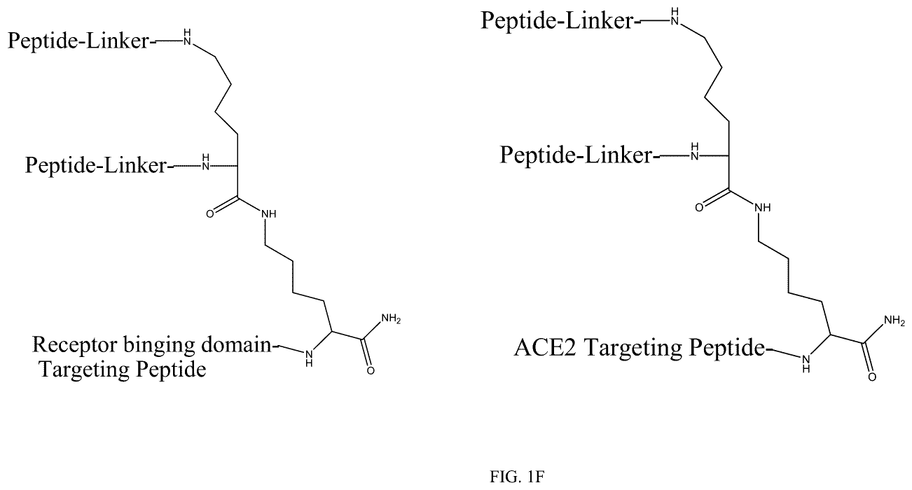

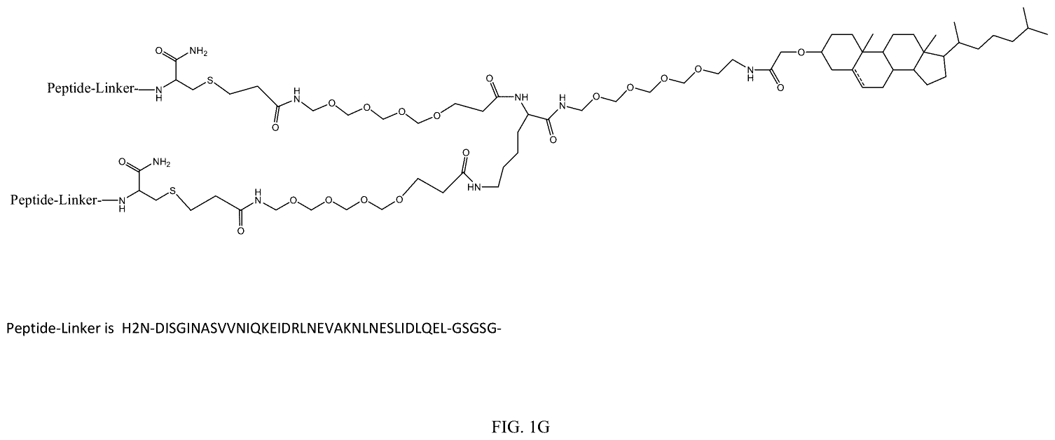

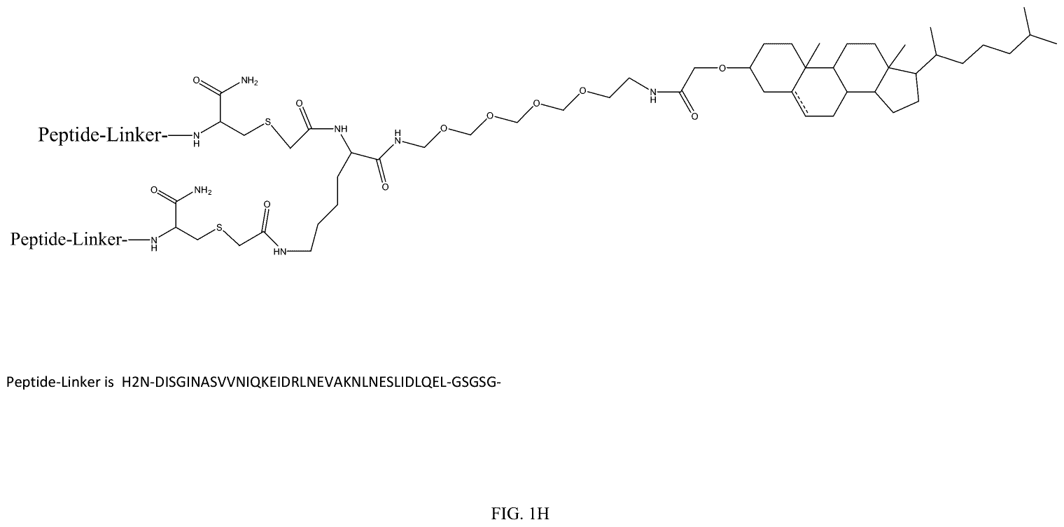

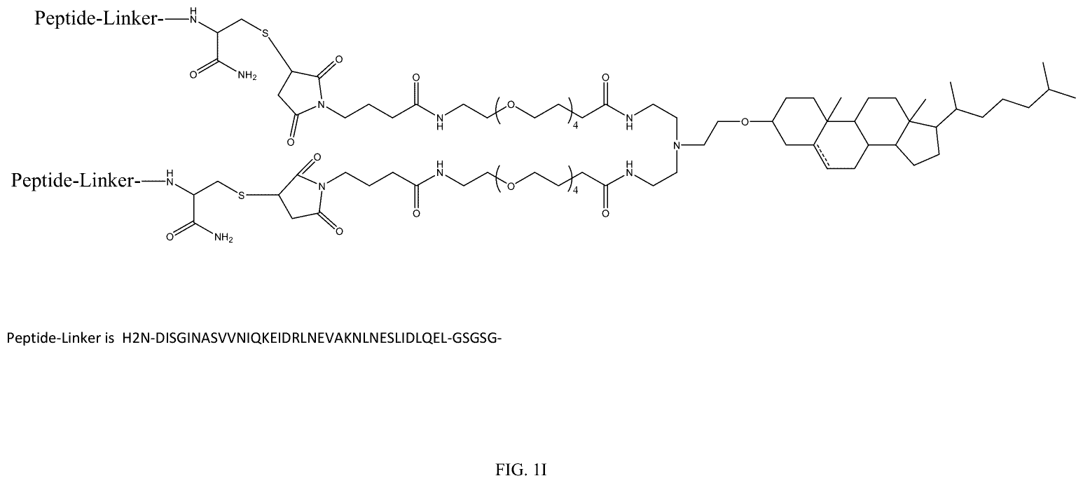

In embodiments, a compound of the invention has the structure as shown in FIG. 1H (also referred to as “Decoy 101” or “DCOY101”).

In embodiments, a compound of the invention has the structure as shown in FIG. 1I (also referred to as “Decoy 102” or “DCOY102”).

In embodiments, a compound of the invention has the structure as shown in FIG. 1G (also referred to as “Decoy 103” or “DCOY103”).

The linkers of DCOY102 and DCOY103 new linkers provide improved stability over DCOY101. Additionally, DCOY102 and DCOY103 show improved solubility in PBS buffer are below: Decoy 101: ˜0.04 mg/mL=0.004 mM; Decoy 102: ˜0.35 mg/mL=0.035 mM, Decoy 103: ˜0.19 mg/mL=0.018 mM.

The compositions of the invention comprise a compound as described herein and a pharmaceutically acceptable carrier. For example, the composition can be administered systemically or locally. The composition can be administered for oral, intravenous, intramuscular, rectal, cutaneous, subcutaneous, topical, transdermal, sublingual, nasal, inhalation, or vaginal delivery, for example. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols. The compositions may be formulated according to conventional pharmaceutical practice (see, e.g., Remington: The Science and Practice of Pharmacy, 22^{nd} edition, 2013, ed. L. V. Allen, Pharmaceutical Press, Philadelphia, and Encyclopedia of Pharmaceutical Technology, 4.sup.th Edition, ed. J. Swarbrick, 2013, CRC Press, New York).

Compounds may be formulated in a variety of ways that are known in the art. For example, one or more compounds of the invention and any additional biologically active agent, if present, as defined herein may be formulated together or separately.

Each compound of the invention, alone or in combination with one or more active agents as described herein, can be formulated for controlled release (e.g., sustained or measured) administration, as described in U.S. Patent Application Publication Nos. 2003/0152637 and 2005/0025765, each incorporated herein by reference. For example, a compound of the invention, alone or in combination with one or more of the biologically active agents as described herein, can be incorporated into a capsule or tablet that is administered to the patient.

Controlled release formulations known in the art include specially coated pellets, polymer formulations or matrices for surgical insertion or as sustained release microparticles, e.g., microspheres or microcapsules, for implantation, insertion, infusion or injection, wherein the slow release of the active medicament is brought about through sustained or controlled diffusion out of the matrix and/or selective breakdown of the coating of the preparation or selective breakdown of a polymer matrix. Other formulations or vehicles for controlled, sustained or immediate delivery of an agent to a preferred localized site in a patient include, e.g., suspensions, emulsions, gels, liposomes and any other suitable art known delivery vehicle or formulation acceptable for subcutaneous or intramuscular administration.

Suitable biocompatible polymers can be utilized as the controlled release material. The polymeric material may comprise biocompatible, biodegradable polymers, and, in certain preferred embodiments, is preferably a copolymer of lactic and glycolic acid. Preferred controlled release materials which are useful in the formulations of the invention include the polyanhydrides, polyesters, co-polymers of lactic acid and glycolic acid (preferably wherein the weight ratio of lactic acid to glycolic acid is no more than 4:1 i.e., 80% or less lactic acid to 20% or more glycolic acid by weight) and polyorthoesters containing a catalyst or degradation enhancing compound, for example, containing at least 1% by weight anhydride catalyst such as maleic anhydride. Examples of polyesters include polylactic acid, polyglycolic acid and polylactic acid-polyglycolic acid copolymers. Other useful polymers include protein polymers such as collagen, gelatin, fibrin and fibrinogen and polysaccharides such as hyaluronic acid.

In additional embodiments, the controlled release material, which in effect acts as a carrier for a compound of the invention can further include a bioadhesive polymer such as pectins (polygalacturonic acid), mucopolysaccharides (hyaluronic acid, mucin) or non-toxic lectins or the polymer itself may be bioadhesive, e.g., polyanhydride or polysaccharides such as chitosan. In embodiments where the biodegradable polymer comprises a gel, one such useful polymer is a thermally gelling polymer, e.g., polyethylene oxide, polypropylene oxide (PEO-PPO) block copolymer such as PLURONIC.TM. F127 from BASF Wyandotte.

Formulations for oral use include tablets containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, taste masking agents (such as hydroxypropyl methylcellulose, hydroxypropyl cellulose) and the like.

One or more compounds of the invention may be mixed together in a tablet, capsule, or other vehicle, or may be partitioned. In one example, a compound of the invention is contained on the inside of the tablet, and the biologically active agent is on the outside of the tablet, such that a substantial portion of the biologically active agent is released prior to the release of the compound of the invention.

Formulations for oral use may also be provided as chewable tablets, or as hard gelatin capsules wherein the active ingredient(s) are mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment. Formulations to the mouth may also be provided as a mouthwash, an oral spray, oral rinse solution, or oral ointment, or oral gel.

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

Liquid forms in which the compounds and compositions of the present invention can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Formulations suitable for parenteral administration (e.g., by injection), include aqueous or non-aqueous, isotonic, pyrogen-free, sterile liquids (e.g., solutions, suspensions), in which the compound is dissolved, suspended, or otherwise provided (e.g., in a liposome or other microparticulate). Such liquids may additional contain other pharmaceutically acceptable ingredients, such as anti-oxidants, buffers, preservatives, stabilizers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection. Typically, the concentration of the compound in the liquid is from about 1 ng/ml to about 10 ug/ml, for example from about 10 ng/ml to about 1 ug/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets.

The composition of the invention can comprise a liquid vehicle which is suitable for nasal administration. The vehicle is preferably an aqueous solution. More preferably, the vehicle is an aqueous solution which includes a viscosity enhancing agent and, optionally one or more additional excipients which, for example, improve formulation stability and/or comfort upon administration.

A variety of viscosity enhancing agents are known in the art. Viscosity enhancing agents include hydrophilic polymers, such as polysaccharides, polysaccharide derivatives, proteins and synthetic polymers. Examples include, but are not limited to, acacia, tragacanth, alginic acid, carrageenan, locust bean gum, guar gum, gelatin, hyaluronic acid, polyacrylate, polyacrylate/alkylacrylate copolymers, polyvinyl alcohol, polyvinylpyrrolidone, starch, propylene glycol alginate, maltodextrin, and cellulose ether derivatives, such as methylcellulose, hydroxyethylcellulose, hydroxypropylmethylcellulose, hydroxypropylcellulose, and carboxymethylcellulose. Where possible, salt forms of any of the foregoing are preferred. Preferred viscosity enhancing agents include hyaluronic acid, including sodium hyaluronate; carboxymethylcellulose, including sodium carboxymethylcellulose and calcium carboxymethylcellulose; methylcellulose, hydroxyethylcellulose, hydroxypropylmethylcellulose, and hydroxypropylcellulose.

The composition optionally includes one or more additional excipients which, for example, increase the ease of administration, the comfort of the subject, or the stability of the composition. Suitable additional excipients include, but are not limited to, tonicity modifiers, such as sodium chloride and dextrose; antioxidants, such as butylated hydroxyanisole; buffers, such as sodium bicarbonate, sodium citrate and sodium phosphate; preservatives, such as benzalkonium chloride, ethanol, propylene glycol, benzoyl alcohol, phenethyl alcohol, chlorobutanol or methylparaben; pH adjusters, such as hydrochloric acid, sulfuric acid and sodium hydroxide; surfactants, such as Polysorbate 80, Polysorbate 20, and polyoxyl 400 stearate; chelating agents, such as disodium EDTA; antioxidants; co-solvents, such as ethanol, PEG 400, and propylene glycol; penetration enhancers, such as oleic acid; and humectants, such as glycerin (see S. Thorat, Sch. J. App. Med. Sci. 2016, 4(8D):2976-2985; D. Marx et al., IntechOpen, DOI: 10.5772/59468. Available from: intechopen.com/books/drug-discovery-and-development-from-molecules-to-medicine/intranasal-drug-administration-an-attractive-delivery-route-for-some-drugs).

In one embodiment, the vehicle consists of sodium hyaluronate, aloe vera, allantoin, sodium chloride, sodium bicarbonate, glycerin, propylene glycol, propylene glycol, benzalkonium chloride and USP grade purified water. A suitable vehicle is sold by NEILMED™ under the tradename NASOGEL™.

The amount of active agent in the composition can vary, for example, from about 0.5% by weight to about 25% by weight.

The pH of the formulation is tolerable in the nasal cavity and preferably at least about 8.0. Buffers that can be used in the formulation include, but are not limited to phosphate, TRIS, [tris(hydroxymethyl) methylamino] propanesulfonic acid, 2-(bis(2-hydroxyethyl)amino)acetic acid, and N-[tris(hydroxymethyl)methyl]glycine, and Alkaline Buffer (Seachem).

A pharmaceutical composition suitable for nasal or pulmonary administration comprising a water soluble solvent selected from the group consisting of propylene glycol, glycerin, polyethylene glycol, and combinations thereof. The composition can further comprise one or more of a polysaccharide gum, a non-ionic surfactant, and a preservative. An exemplary polysaccharide gum is sclerotium gum. Exemplary surfactants are poloxamers, including, but not limited, to poloxamer 188. The preservative can, for example, be benzalkonium chloride.

The composition can be a dry powder and delivered by a dry powder inhaler, suspended in a propellant or in an aqueous suspension or solution and delivered via a nebulizer.

For example, a solution or suspension of the active agent and a pulmonary excipient, such as lactose, can be spray dried to form particles having a fine particle fraction sufficient to deliver to the lungs or upper respiratory system. Alternatively, an aqueous solution or suspension can be sonicated, thereby aerosolizing the solution/suspension to a droplet size that can be inhaled, e.g., via a nebulizer.

Excipients include carbohydrates including monosaccharides, disaccharides and polysaccharides. For example, monosaccharides such as dextrose (anhydrous and monohydrate), galactose, mannitol, D-mannose, sorbitol, sorbose and the like; disaccharides such as lactose, maltose, sucrose, trehalose, and the like; trisaccharides such as raffinose and the like; and other carbohydrates such as starches (hydroxyethylstarch), cyclodextrins and maltodextrins. Other excipients suitable for use with the present invention, including amino acids, are known in the art such as those disclosed in WO 95/31479, WO 96/32096, and WO 96/32149. Mixtures of carbohydrates and amino acids are further held to be within the scope of the present invention. The inclusion of both inorganic (e.g., sodium chloride, etc.), organic acids and their salts (e.g., carboxylic acids and their salts such as sodium citrate, sodium ascorbate, magnesium gluconate, sodium gluconate, tromethamine hydrochloride, etc.) and buffers is also contemplated.

The compositions may be used in the form of dry powders or in the form of stabilized dispersions comprising a non-aqueous phase. Accordingly, the dispersions or powders of the present invention may be used in conjunction with metered dose inhalers (MDIs), dry powder inhalers (DPIs), atomizers, nebulizers or liquid dose instillation (LDI) techniques to provide for effective drug delivery. With respect to inhalation therapies, those skilled in the art will appreciate that the hollow and porous microparticles of the present invention are particularly useful in DPIs. Conventional DPIs comprise powdered formulations and devices where a predetermined dose of medicament, either alone or in a blend with lactose carrier particles, is delivered as an aerosol of dry powder for inhalation.

The medicament is formulated in a way such that it readily disperses into discrete particles with a mass median aerodynamic diameters of the powders will characteristically range from about 0.5-10, preferably from about 0.5-5.0 microns MMAD.

As discussed above, the stabilized dispersions disclosed herein may also be administered to the nasal or pulmonary air passages of a patient via aerosolization, such as with a metered dose inhaler. MDIs are well known in the art and could easily be employed for administration of the claimed dispersions without undue experimentation. Breath activated MDIs, as well as those comprising other types of improvements which have been, or will be, developed are also compatible with the stabilized dispersions and present invention and, as such, are contemplated as being within the scope thereof. However, it should be emphasized that, in preferred embodiments, the stabilized dispersions may be administered with an MDI using a number of different routes including, but not limited to, topical, nasal, pulmonary or oral. Those skilled in the art will appreciate that, such routes are well known and that the dosing and administration procedures may be easily derived for the stabilized dispersions of the present invention.

Along with the aforementioned embodiments, the stabilized dispersions of the present invention may also be used in conjunction with nebulizers as disclosed in PCT WO 99/16420, the disclosure of which is hereby incorporated in its entirety by reference, in order to provide an aerosolized medicament that may be administered to the pulmonary air passages of a patient in need thereof. Nebulizers are well known in the art and could easily be employed for administration of the claimed dispersions without undue experimentation. Breath activated nebulizers, as well as those comprising other types of improvements which have been, or will be, developed are also compatible with the stabilized dispersions and present invention and are contemplated as being within the scope thereof.

Along with DPIs, MDIs and nebulizers, it will be appreciated that the stabilized dispersions of the present invention may be used in conjunction with liquid dose instillation or LDI techniques as disclosed in, for example, WO 99/16421 hereby incorporated in its entirety by reference. Liquid dose instillation involves the direct administration of a stabilized dispersion to the lung. In this regard, direct pulmonary administration of bioactive compounds is particularly effective in the treatment of disorders especially where poor vascular circulation of diseased portions of a lung reduces the effectiveness of intravenous drug delivery. With respect to LDI the stabilized dispersions are preferably used in conjunction with partial liquid ventilation or total liquid ventilation. Moreover, the present invention may further comprise introducing a therapeutically beneficial amount of a physiologically acceptable gas (such as nitric oxide or oxygen) into the pharmaceutical microdispersion prior to, during or following administration.

### Methods of Use

The invention also includes methods of using the composition of the invention for treating or preventing an infection in a subject in need thereof. The method comprises the step of administering an effective amount of the composition to the subject. The infection can be an infection of the gastrointestinal tract or upper or lower respiratory tract, including the common cold, influenza, respiratory syncytial virus infection, Severe Acute Respiratory Syndrome, Middle East Respiratory Syndrome, COVID-19 or a disease caused by another emerging zoonotic virus, such as a zoonotic coronavirus. In specific aspects, the methods of the invention treat a viral respiratory infection, such as a SARS-CoV-2 (COVID-19) respiratory infection.

In an embodiment, the invention provides a method for treating a respiratory infection caused by a SAR-Cov-2 variant in a subject in need thereof. In embodiments, the method comprises administering to the subject an effective amount of a compound having the formula: (Peptide-Linker)ₙ-B-Hydrophobic Moiety wherein each Peptide is independently a HRC Peptide or a targeting peptide, provided that at least one peptide is a HRC Peptide, each Linker is independently a bivalent linking moiety, B is a multivalent moiety comprising cysteine, X, and optionally Y, and/or optionally Z, wherein X, Y and Z are defined herein, and n is an integer selected from 1, 2, 3 or more; and wherein the variant comprises at least 5 mutations wherein the at least 5 mutations are independently in the spike protein S1 subunit or the S2 subunit or combinations thereof.

In embodiments, the compound comprises a targeting peptide and one or more HRC Peptide. In embodiments, the targeting peptide is an ACE2 targeting peptide or a receptor binding domain peptide. In embodiments, the Hydrophobic Moiety is cholesterol.

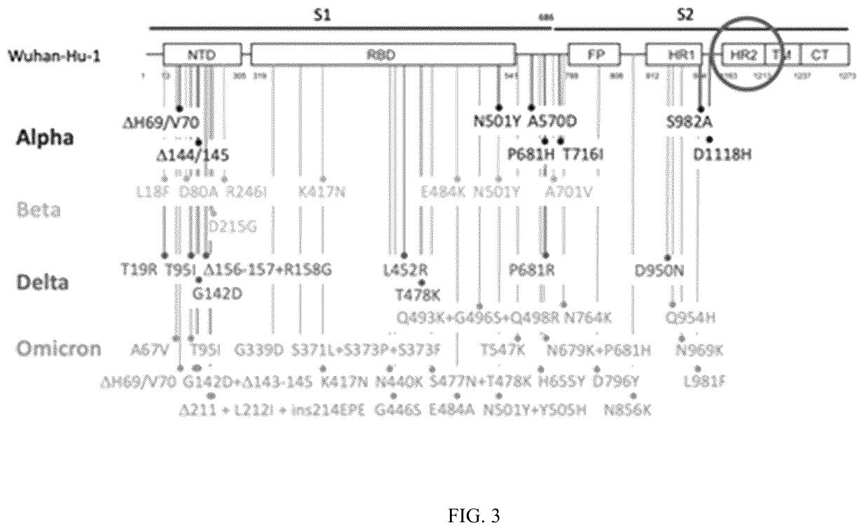

In embodiments, the at least 5 mutations are independently in N-Terminal domain (NTD), the receptor binding domain (RBD), the fusion peptide (FP)domain, the heptad repeat 1 (HR1) domain, or combinations thereof. In embodiments, the at least 5 mutations are independently selected from the mutations listed in FIG. 3.

In embodiments, the at least 5 mutations are independently selected from at least 5 mutations from the SAR-Cov-2 Alpha variant; at least 5 mutations from the SAR-Cov-2 Beta variant; at least 5 mutations from the SAR-Cov-2 Delta variant; or at least 5 mutations from the SAR-Cov-2 Omicron variant. In embodiments, the at least 5 mutations are independently selected from at least 5 mutations from the SAR-Cov-2 Alpha variant. In embodiments, the at least 5 mutations are independently selected from at least 5 mutations from the SAR-Cov-2 Beta variant. In embodiments, the at least 5 mutations are independently selected from at least 5 mutations from the SAR-Cov-2 Delta variant. In embodiments, the at least 5 mutations are independently selected from at least 5 mutations from the SAR-Cov-2 Omicron variant.

In embodiments, the variant comprises at least 10 mutations. In embodiments, the variant comprises at least 15 mutations. In embodiments, the variant comprises at least 20 mutations.

In embodiments, the SARS-CoV-2 variant comprises at least one variant selected from B.1.1.7 (Alpha), B.1.351 (Beta), P.1 (Gamma), B.1.617.2 (Delta), B.1.429/B.1.427 (Epsilon), B.1.617.1 (Kappa), B.1.525 (Eta), B.1.526 (Iota), P.3 (Theta), P.2 (Zeta), and B.1.1.529 (Omicron).

In embodiments, the SARS-CoV-2 variant comprises at least one variant selected from A.1-A.6, B.3-B.7, B.9, B.10, B.13-B.16, B.2, B.1 lineage, P.1, P.2, P.3, and R.1.

In embodiments, the B.1 lineage comprises at least one of (including, but not limited to, B.1, B.1.1, B.1.1.7, B.1.1.7 with E484K, B.1.2, B.1.5-B.1.72, B.1.9, B.1.13, B.1.22, B.1.26, B.1.37, B.1.3-B.1.66, B.1.177, B.1.243, B.1.313, B.1.351, B.1.427, B.1.429, B.1.525, B.1.526, B.1.526.1, B.1.526.2, B.1.617, B.1.617.1, B.1.617.2, B.1.617.3, B.1.619, B.1.620, and B.1.621.

In embodiments, the administration is achieved using an intranasal spray, an inhaler or a nebulizer.

In embodiments, the compound is administered in combination with at least one other antiviral active agent or therapy.

The subject, preferably a human, can be an individual diagnosed with the infection and is either symptomatic, pre-symptomatic, or asymptomatic, or at risk for developing infection. For example, the subject can be at risk for developing the viral respiratory infection due to direct or indirect exposure or possible exposure to the virus (such as SARS-CoV-2 or a mutant thereof), such as via exposure to an infected individual or a virus-contaminated fomite. The subject can be a resident of, or a visitor to, a community in which the viral respiratory infection has been identified, for example, the subject can be a family member of an infected individual or the subject can work in a health care setting caring for infected individuals. In certain embodiments, the subject at risk for infection is asymptomatic and has tested negative for presence of the virus prior to the commencement of therapy. In specific examples, the subject can be at risk for developing COVID-19 due to exposure to the SARS-CoV-2 virus, for example, from the respiratory droplets or aerosols of an infected individual and/or contact with a contaminated fomite. In yet further aspects, the subject is suffering from COVID-19 including subjects suffering from mild, moderate or severe COVID-19.

In certain embodiments of the method of the invention, the subject suffers from another disease or condition, such as chronic obstructive pulmonary disease (COPD) or ulcerative colitis, which can be exacerbated by an infection.

The composition is preferably administered to the subject before the subject is symptomatic (e.g., pre-symptomatic), or at the onset of symptoms. The composition can be administered at a variety of dosing schedules. For example, the composition can be administered one or more times and over a course of one or more days. In certain embodiments, the composition is administered one or more times per day for one to 10 days. In certain embodiments, the composition is administered one or more times per day until the subject is asymptomatic and/or testing for the virus is negative.

The composition can be administered to the nasal passages using routine methods and devices (see D. Marx et al., IntechOpen, DOI: 10.5772/59468. Available from: https://www.intechopen.com/books/drug-discovery-and-development-from-molecules-to-medicine/intranasal-drug-administration-an-attractive-delivery-route-for-some-drugs). For example, the composition can be administered to the nasal passages as drops or as an aerosol spray, for example, using an aerosol bottle or a multi-dose spray pump, which can provide a uniform metered dose. The volume per dose can be varied, but is typically from about 50 to about 150 μl. The desired volume will depend on the desired dose of the active agent and the concentration of the active agent in the composition.

Where delivery to the pulmonary system, or lungs, is desired it can be efficacious to aerosolize a low concentration solution of the active agent for an extended period, such as overnight.

### Combination Therapies

The compound or composition described herein can be co-administered with other active agents and therapies.

In embodiments, the other active agent includes, but is not limited to, antibodies against SARS-CoV-2. Suitable antibodies are described in, for example, US 2022/0017604, US 2022/0017614; US 2021/0403550, US 2021/0395345, US 2021/0403537; US 2021/0388066, US 2021/0388065, US 2021/0347859, or US 2021/0309733, which are incorporated herein by reference. In embodiments, the antibody is a monoclonal antibody such as casirivimab, imdevimab, bamlanivimab, or etesevimab. In embodiments, the antibody is a monoclonal antibody therapy such as casirivimab plus imdevimab, bamlanivimab, or bamlanivimab plus etesevimab.

The active agents and compositions of the present invention are also intended for use with general care provided patients with viral infections, including parenteral fluids (including dextrose saline and Ringer's lactate) and nutrition, antibiotic (including metronidazole and cephalosporin antibiotics, such as ceftriaxone and cefuroxime) and/or antiviral prophylaxis, fever (e.g., acetaminophen) and pain medication, antiemetic (such as metoclopramide) and/or antidiarrheal agents, vitamin and mineral supplements (including Vitamin K and zinc sulfate), anti-inflammatory agents (such as ibuprofen), pain medications, and medications for other common diseases in the patient population, such as artemether, artesunate-lumefantrine combination therapy), quinolone antibiotics, such as ciprofloxacin, macrolide antibiotics, such as azithromycin, cephalosporin antibiotics, such as ceftriaxone, or aminopenicillins, such as ampicillin), or shigellosis.

The combination therapy may be administered as a simultaneous or sequential regimen. When administered sequentially, the combination may be administered in two or more administrations.

Co-administration of a compound of the invention with one or more other active therapeutic agents generally refers to simultaneous or sequential administration of a compound of the invention and one or more other active therapeutic agents, such that therapeutically effective amounts of the compound of the invention and one or more other active therapeutic agents are both present in the body of the patient.

Co-administration includes administration of unit dosages of the compounds of the invention before or after administration of unit dosages of one or more other active therapeutic agents, for example, administration of the compounds of the invention within seconds, minutes, or hours of the administration of one or more other active therapeutic agents and/or as part of the same treatment regimen. For example, a unit dose of a compound of the invention can be administered first, followed within seconds or minutes or days by administration of a unit dose of one or more other active therapeutic agents. Alternatively, a unit dose of one or more other therapeutic agents can be administered first, followed by administration of a unit dose of a compound of the invention within seconds or minutes or days. In some cases, it may be desirable to administer a unit dose of a compound of the invention first, followed, after a period of hours (e.g., 1-12 hours), by administration of a unit dose of one or more other active therapeutic agents. In other cases, it may be desirable to administer a unit dose of one or more other active therapeutic agents first, followed, after a period of hours (e.g., 1-12 hours), by administration of a unit dose of a compound of the invention.

The combination therapy may provide “synergy” and “synergistic,” i.e., the effect achieved when the active ingredients used together is greater than the sum of the effects that results from using the compounds separately.

As used herein, the words “a” and “an” are meant to include one or more unless otherwise specified. For example, the term “an agent” encompasses both a single agent and a combination of two or more agents.

The term “treating” or “treatment” as used herein covers the treatment of the disease or condition of interest (e.g., a respiratory infection) in a mammal, preferably a human, having the disease or condition of interest, and includes, for example: preventing or delaying the onset of the disease or condition from occurring in a mammal, in particular, when such mammal is at risk of developing the disease but has not yet become symptomatic and/or been diagnosed as having it; inhibiting the disease or condition, i.e., arresting its development; relieving the disease or condition, i.e., causing regression of the disease or condition; and/or stabilizing the disease or condition. Treatment includes ameliorating or lessening the severity of symptoms of the disease or condition, and/or inhibition of further progression or worsening of those symptoms. Treatment also includes shortening the time course and/or severity of a disease or condition compared to the expected or historical time course and/or severity of the disease.

As used herein the terms “preventing,” means causing the clinical symptoms of a disease or condition not to develop and includes inhibiting the onset of a viral infection in a subject that may be exposed to or predisposed to the viral infection but does not yet experience or display symptoms of the infection.

An “effective amount” or a “therapeutically effective amount” of a compound or composition described herein refers to an amount of the compound that is sufficient to achieve a specific effect or result, and/or prevents or treats the disease or condition and/or the symptoms therefore, for example, alleviating, in whole or in part, symptoms associated with the disorder or condition, or halts or slows further progression or worsening of those symptoms, or prevents or provides prophylaxis for the disorder or condition. The “effective amount” and “therapeutically effective amount” includes specifically an anti-viral amount of a compound of the invention (alone or in combination with another active agent) or the composition described herein.

While this invention has been particularly shown and described with references to preferred embodiments thereof, it will be understood by those skilled in the art that various changes in form and details may be made therein without departing from the scope of the invention encompassed by the appended claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference. The relevant teachings of all patents, published applications and references cited herein are incorporated by reference in their entirety.

### Example 1

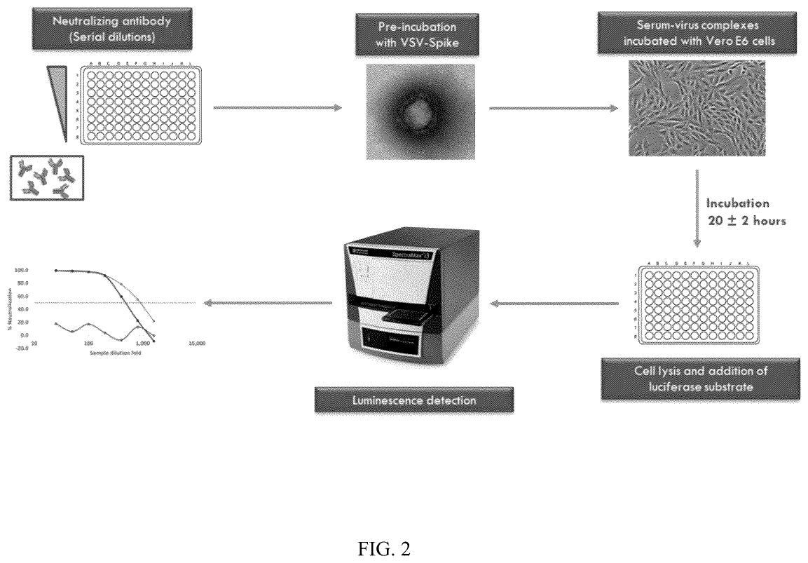

A compound was tested using an optimized SARS-CoV-2 pseudotyped virus neutralization assay (PNA). This pseudotyped virus neutralization assay is used to quantify the titer of neutralizing antibody or the half maximal inhibitory concentration (IC50) of molecules for SARS-CoV-2 (FIG. 2). Pseudotyped virus particles are made using a genetically modified Vesicular Stomatitis Virus from which the glycoprotein G was removed (VSVΔG). The VSVΔG virus is transduced in HEK293T cells previously transfected with the spike glycoprotein of different SARS-CoV-2 coronavirus strains (WT (Wuhan), Alpha (B.1.1.7, UK), Beta (B.1.351, SA) or Gamma (P.1, BRA) strains), for which the last 19 amino acids of the cytoplasmic tail have been removed (ΔCT). The pseudoparticles generated (VSVΔG-Spike ΔCT) contain a luciferase reporter which can be quantified in relative luminescence units (RLU).

Neutralizing activity against SARS-CoV-2 pseudovirus was measured using a single-round infection assay in 293T/ACE2 target cells. Pseudotyped virus particles were produced in 293T/17 cells (ATCC) by co-transfection of plasmids encoding codon-optimized SARS-CoV-2 full-length Spike (containing G at position 614), packaging plasmid pCMV R8.2, and luciferase reporter plasmid pHR′ CMV-Luc. Wildtype and B.1.1.529 (Omicron) variant spike, packaging and luciferase plasmids were kindly provided by Dr. Barney Graham (NIH, Vaccine Research Center). B.1.351 (Beta) and B.1.617.2 (Delta) variant Spike plasmids were provided by Dr. Bing Chen (Children's Hospital, Boston MA). The 293T cell line stably overexpressing the human ACE2 cell surface receptor protein was kindly provided by Drs. Michael Farzan and Huihui Ma (The Scripps Research Institute). For neutralization assays, serial dilutions of peptides were performed in duplicate followed by addition of pseudovirus. Pooled serum samples from convalescent COVID-19 patients or PBS/10% DMSO diluent buffer were used as positive and negative controls, respectively. Plates were incubated for 1 hour at 37° C. followed by addition of 293/ACE2 target cells (1×10⁴/well). Wells containing cells+pseudovirus (without sample) or cells alone acted as positive and negative infection controls, respectively. Assays were harvested on day 3 using Promega BrightGlo luciferase reagent and luminescence detected with a Promega GloMax luminometer. Titers are reported as the concentration of peptide that inhibited 50% or 80% virus infection (IC₅₀ and IC₈₀ titers, respectively). All neutralization experiments were repeated twice with similar results.

The pseudotyped virus neutralization assay was used to assess the inhibition capacity of a compound of the invention against four strains of SARS-CoV-2 (WT, Alpha, Beta and Gamma). The compound was tested at a starting concentration of 5 μM and was serially diluted in 96-well plates, following three different serial dilution schemes: 10-fold, 5-fold and 3-fold dilutions. As the compound was first re-suspended in DMSO, a 3-fold serial dilution of a DMSO solution (starting concentration 0.052% DMSO) was also tested in PNA with the same strains in order to address the compound's diluent effect on the assay. A pre-determined amount of each pseudotyped virus (corresponding to approximately 1,200,000 RLU/well) was applied to the plates and incubated with the diluted compound to allow binding of the compound to the pseudotyped viruses. A positive control for the assay was also included on each plate and tested with all four strains of Spike pseudotyped viruses. This control consists of a human serum with neutralizing antibodies for SARS-CoV-2 Spike protein that is routinely used in PNA testing.

After the incubation of the compound/serum-pseudotyped virus complexes, media was removed from 96-well plates of Vero E6 cells and the compound-pseudotyped virus complexes were transferred on the cells. Test plates were incubated at 37° C. with 5% CO2 overnight. Following incubation, luciferase substrate was added to the VeroE6 cells and plates were read using a plate reader detecting luminescence. The intensity of the light being emitted is inversely proportional to the level of neutralizing potency of the compound. The compound IC50 were then calculated for all tested dilutions of the Wuhan and Spike variants using a standard 4-parameter logistic model. The half maximal inhibitory concentration (IC50) calculated for the 10-fold serial dilution and the corresponding R square values are presented in Table 1. The IC50 for the EK1 positive control vs. WT, omicron, delta, and beta were 0.622 μm, 0.241 μm, 0.244 μm, and 0.527 μm, respectively (data not shown).

TABLE 1

| | Spike | | |
|---|---|---|---|
| Virus strain | Mutation | IC50 | R² |
| Example 1 | | | |
| WT (Wuhan) | | 0.046 μM | 0.986 |
| Alpha | 8 | 0.057 μM | 0.987 |
| Beta | 9 | 0.051 μM | 0.905 |
| Gamma | 10 | 0.060 μM | 0.985 |
| Example 2 | | | |
| Wild type (G614) | | 0.026 μM | |
| Omicron | 30 | 0.014 μM | |
| Beta | 9 | 0.009 μM | |
| Delta | 10 | 0.009 μM | |

| Term/Abbreviation | Definition |
|---|---|
| % | Percentage |
| BRA | Brazil |
| CC | Cell Control |
| CoV | Coronavirus |
| CT | Cytoplasmic Tail |
| CV | Coefficient of Variation |
| DMSO | Dimethyl Sulfoxide |
| G | Glycoprotein G |
| GMC | Geometric Concentration |
| GxP | Good x Practices |
| HEK | Human Embryonic Kidney |
| ID | Identification |
| IC50 | Half maximal inhibitory concentration |
| Log | Logarithm |
| PC | Pseudoparticle Control |
| PNA | Pseudotyped virus Neutralization Assay |
| RBD | Receptor Binding Domain |
| RLU | Relative Luminescence Units |
| SA | South-Africa |
| SARS-CoV-2 | Severe Acute Respiratory Syndrome |
| | Coronavirus 2 |
| SD | Standard Deviation |
| SMP | SoftMax Pro |
| Sple | Sample |
| SQRT | Square Root |
| STD | Standard |
| TO | Task Order |
| UK | United Kingdom |
| VSV | Vesicular Stomatitis Virus |

### Example 2—Efficacy of Potential Antiviral Therapeutics Against SARS-CoV-2 in Hamster

The objective of this study was to test the in vivo efficacy of DCOY101 as a potential antiviral therapeutic against SARS-CoV-2, the causative agent of COVID-19, in the hamster model. Test materials (TA), i.e., DCOY101, solutions and dosing are defined in Table 3. Bulk test materials stored at approximately −20±5° C. and protected from light, until used. The dosing formulations of the test article were prepared at the beginning of the dosing and aliquoted and stored at −20±5 TC.

TABLE 3
Test Article Preparation

| | | | TA prep. | |
|---|---|---|---|---|
| Solution | | Dose | Solution | |
| No. | ID of TA | (mg/kg) | mg/ml | Vehicle |
| 1 | Vehicle | 0 | 0 | 20% PEG300* |
| 2 | DCOY101-10 | 10 | 20 | in 1x PBS |
| 3 | DCOY101-5 | 5 | 10 | |
| 4 | DCOY101-2.5 | 2.5 | 5 | |
| 5 | DCOY101-1 | 1 | 2 | |

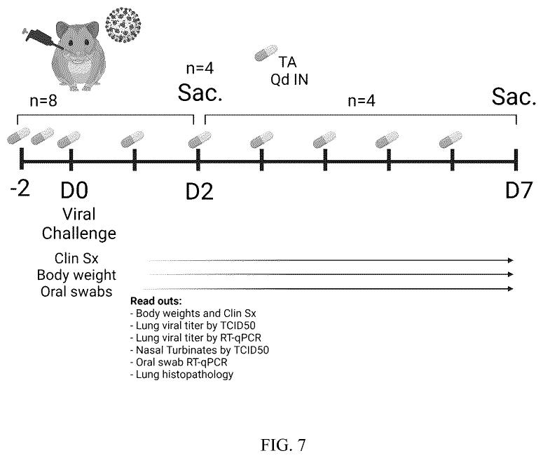

Hamsters were administered intranasally vehicle or test material dosing formulations once a day (Q.d.) for 9 days (from Study Day −2 to Day 6) according to the experimental design (FIG. 7) and detailed in Table 2. On Study Day 0, hamsters were intranasally dose targeting 5×10⁴ TCID₅₀/animal of the B.1.617.2 of SARS-CoV-2 and 4±2 hours later treated with TA, according to the following experimental design (FIG. 7) and detailed in Table 2.

TABLE 2
Experimental Design

| | | | Dosing | | | Int. | Terminal |
|---|---|---|---|---|---|---|---|
| | | | Schedule | | Oral | Sac. | Sac. |
| ID | N | Treatment | Qd | Route | Swabs | (n = 4) | (n = 4) |
| G1 | 8 | Vehicle Control | −2 to d6 | IN | d1-d7 | d2 | d7 |
| G2 | 8 | DCOY101 - 10 | −2 to d6 | IN | d1-d7 | d2 | d7 |
| G3 | 8 | DCOY101 - 5 | −2 to d6 | IN | d1-d7 | d2 | d7 |
| G4 | 8 | DCOY101 - 2.5 | −2 to d6 | IN | d1-d7 | d2 | d7 |
| G5 | 8 | DCOY101 - 1 | −2 to d6 | IN | d1-d7 | d2 | d7 |

| | |
|---|---|
| Total | 40 + 4* |

Experimental endpoints consisted of moribundity/mortality observations, body weight and body weight change, clinical signs determinations, gross necropsy, viral shedding on oral swabs by RT-qPCR, viral burden in lung tissue and nasal turbinates by TCID₅₀ virus titer, and viral genome copy number in lungs by RT-qPCR and lung histopathology analysis.

No mortality was SARS-CoV-2 disease-associated. However, two animals in the control group were accidental fatalities during dosing.

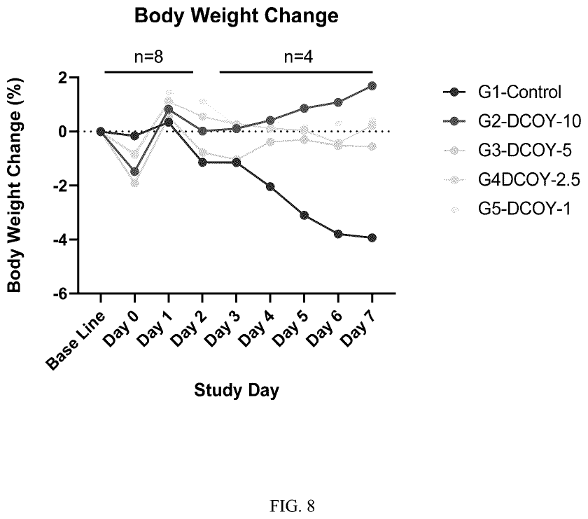

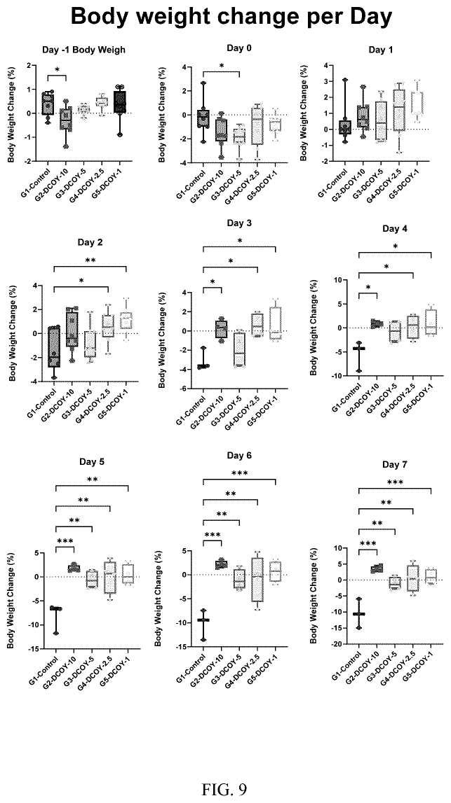

Animals in the control Group 1 designated as vehicle presented an average of −10.5% body weight loss, while all the treatment groups are at a max of −1.1% for Group 3 or even gaining weight+3.4% for Group 2 over the course of the study. FIG. 8. Specifically in Day −2 no differences between any of the groups are shown. On Day −1, Group 2 with the higher dose of 10 mg/kg presents significant (P≤0.0187, *) body weight loss compared to the vehicle control Group 1 (FIG. 9). On Day 0, Group 3, the second higher dose DCOY 5 mg/kg presents significant (P≤0.0315, *) body weight loss compared to the vehicle control Group 1 (FIG. 9). On Day 1, no significant differences were observed in any groups to the administration of the TA at higher doses. From Day 2 to Day 4 slight differences (*) were shown consistently in the groups with the lower doses G4-DCOY-2.5 mg/kg and G5-DCOY-1 mg/kg in this period. During Study Day 5 to Day 7 strong significant differences were observed in a non-conventional drug response concentration. Group 2 the higher dose of DCOY 10 mg/kg (average body weigh change was +3.4%) and the lower dose Group DCOY 1 mg/kg (average body weigh change was +0.9%) have the maximum body weight gain (***, **) when compared to the vehicle control Group 1 (average body weigh change was −10.5%) (FIG. 9).

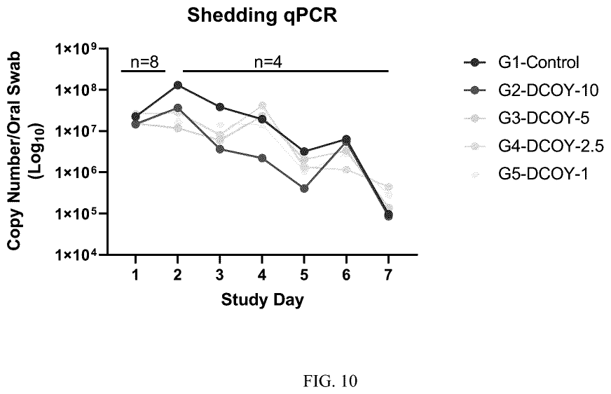

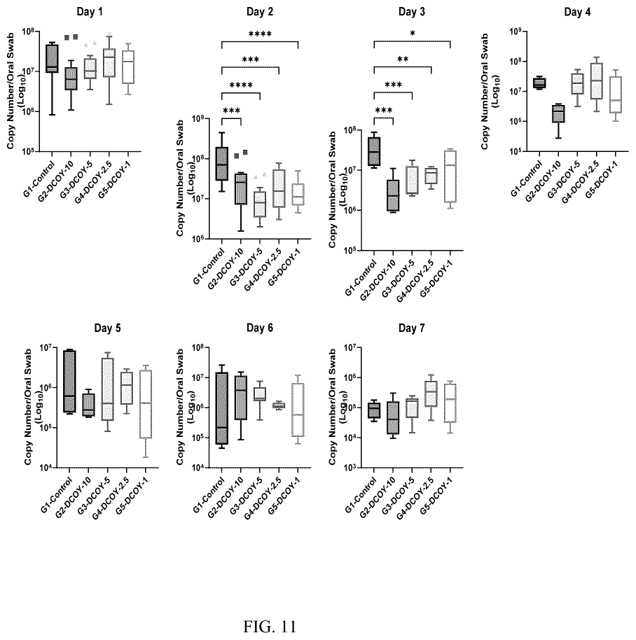

Viral shedding in oral swabs was estimated as viral genome copy number analyzed by RT-qPCR from Day 1 to Day 7. Summary of the shedding profiles are show in FIG. 10. There are no statistical differences in any of the groups in Day 1, 4, 5, 6 or 7. But there is a significant reduction in Day 2 and Day 3. On Day 2 Group 2 showed *** P≤0.001, Group 3**** P≤0.0001, Group 4*** P≤0.001 and Group 5**** P≤0.0001 reduction when compared to the control Group 1. In Day 3, the reduction was not as remarkable for Group 3, Group 4 and Group 5, but Group 2 remained *** P≤0.001. (FIG. 11).

RT-qPCR titers from lung samples show broadly consistent results within each group and follow an inverse correlation with the body weight change. One-step RT-qPCR was performed using isolated RNA from left lung tissue samples and oral swabs to estimate viral copy number. Briefly, RNA was extracted from the lung tissue samples or oral swab supernatants stored in RNA/DNA Shield using the Quick-RNA Viral Kit (Zymo Research). Following the manufacturer's protocol, RT-qPCR analysis was performed using the BlazeTaq Probe One-Step RT-qPCR Kit (GeneCopoeia; Rockville, MD) using isolated RNA as template. The following RT-qPCR cycling conditions were used: 50° C. for 15 minutes (RT), 95° C. for 2 minutes (denature), then 40 cycles of 10 seconds at 95° C. followed by 45 seconds at 62° C. (extension). The following primers and probe were used for SARS-CoV-2 detection:

TABLE 4
qPCR Primers

| | Primer Sequence |
|---|---|
| Primers: | |
| 2019-nCoV_N1-F: | 5′-GACCCCAAAATCAGCGAAAT-3′ |
| 2019-nCoV_N1-R: | 5′-TCTGGTTACTGCCAGTTGAATCTG-3′ |
| Probe: | |
| 2019-nCoV_N1-P: | 5′-FAM-ACCCCGCATTACGTTTGGTGGACC- |
| | BHQ1-3′ |

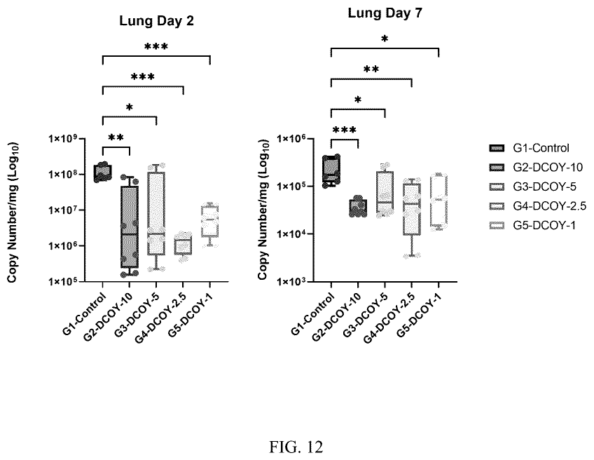

For estimating viral copy number, samples were compared against a standard curve with synthetic RNA. There is more variation in viral load on Day 2 than day 7. However, in both Days 2 and 7, there is a decrease with significant differences (ns P>0.05, * P≤0.05, ** P≤0.01, *** P≤0.001) in Groups 2, 3, 4 and 5 in the lung tissue collected compared to the vehicle control Group 1 as shown in FIG. 12.

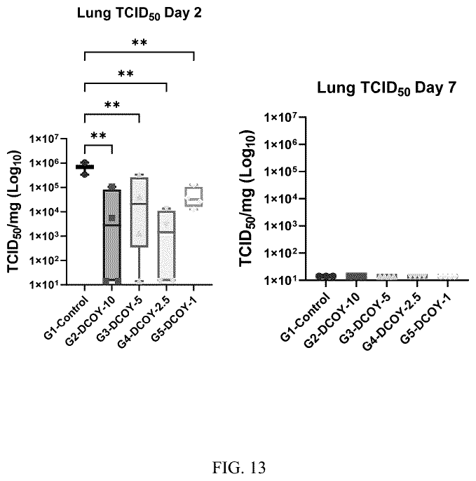

Viral load in lungs was estimated by TCID₅₀ assay on Days 2 and 7. On Day 2 animals in Groups 2, 3, 4 and 5 (** P≤0.01) showed a significant reduction in the viral load accumulation in lungs when compared to the Group 1 (Vehicle Control), but no apparent dose-response. These results are consistent to the RT-qPCR readouts (FIG. 13). On Day 7, no longer infectious virus was detected by the TCID₅₀ assay in any of the groups 1, 2, 3, 4 or 5; however, remaining viral genomes are being still detected by RT-qPCR but in a lower quantity.

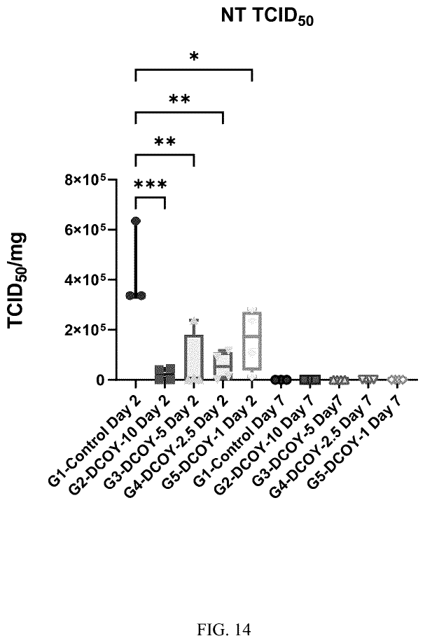

Viral load nasal turbinates in the upper respiratory tract were evaluated by TCID₅₀. The results of this analysis showed that the Vehicle Control Group 1 had higher titers on Day 2 compared to groups 2, 3, 4 or 5, but this difference was not statistically significant. No infectious virus was detected by the TCID₅₀ assay in any of the groups 1, 2, 3, 4 or 5 on Day 7 (FIG. 14).

Based on the reduction of body weight loss consistent with a remarkable reduction in viral load in the lungs of the animals treated with DC101 independently of the dose utilized, the data on this study suggest that DCOY101 has efficacy to reduced SARS-CoV-2 associated phenotype and reduce viral titers in lungs in the peak of the disease and reduced the shedding virus load in the hamster model under the conditions tested.

### Example 3-Pre-Exposure Prophylactic (PrEP) and Post-Exposure Prophylactic (PeP) Efficacy of DCOY101 Against SARS-CoV-2 in the Syrian Hamster Model

The objective of this study was to test the efficacy of DCOY101 when treatment is initiated at different times either pre- or post-infection against SARS-CoV-2, the causative agent of COVID-19, in the hamster model. Test material (TA), i.e., DCOY101, solutions and dosing are defined in Table 6. Bulk test materials were supplied by the Sponsor and stored at approximately −20±5° C. and protected from light, until used. The dosing formulations of the test article were prepared at the beginning of the dosing and aliquoted and stored at −20±5° C.

TABLE 6
Test Article Preparation

| | | | TA prep. | |
|---|---|---|---|---|
| Solution | | Dose | Solution | |
| No. | ID of TA | (mg/kg) | mg/ml | Vehicle |
| 1 | Vehicle | 0 | 0 | 20% PEG300* |
| 2 | DCOY101 | 5 | 10 | in 1x PBS |

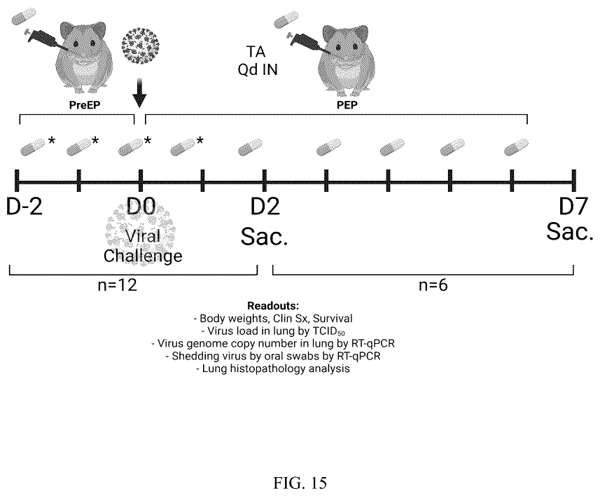

Hamsters were administered intranasally vehicle or test material dosing formulations once a day (Q.d.) for 9 days according to the experimental design (FIG. 15) and detailed in Table 5. On Study Day 0, hamsters were intranasally dose targeting 5×10′ TCID₅₀/animal of the B.1.617.2 of SARS-CoV-2 and 4±2 hours later treated with TA, according to the following experimental design (FIG. 15) and detailed in Table 5.

TABLE 5
Experimental Design

| | | | Dosing | | | Int. | Terminal | |
|---|---|---|---|---|---|---|---|---|
| | | | Schedule | | | Sac. | Sac. | |
| | | | (QD) | | Oral | Day 2 | Day 7 | Animal |
| Group | N | Treatment | START | Route | Swabs | (n = 6) | (n = 6) | Numbers |
| 1 | 12 | Vehicle | −48 hrs to d6 | IN | d1-d7 | Animal # | Animal # | 1-12 |
| | | Control | | | | 7-12 | 1-6 | |
| 2 | 12 | PrEP | −48 hrs to d6 | IN | d1-d7 | Animal # | Animal # | 13-24 |
| | | DCOY101 | | | | 19-24 | 13-18 | |
| 3 | 12 | PEP + 2 | +2 hrs to d6 | IN | d1-d7 | Animal # | Animal # | 25-36 |
| | | DCOY101 | | | | 31-36 | 25-30 | |
| 4 | 12 | PEP + 12 | +12 hrs to d6 | IN | d1-d7 | Animal # | Animal # | 37-48 |
| | | DCOY101 | | | | 43-48 | 37-42 | |
| 5 | 12 | PEP + 24 | +24 hrs to d6 | IN | d1-d7 | Animal # | Animal # | 49-60 |
| | | DCOY101 | | | | 55-60 | 49-54 | |
| 6 | 12 | PEP + 36 | +36 hrs to d6 | IN | d1-d7 | Animal # | Animal # | 61-72 |
| | | DCOY101 | | | | 67-72 | 61-66 | |

Total = 72 + 7* = 79
* 10% (n = 7) more animals will be ordered for randomization process.

Experimental endpoints consisted of moribundity/mortality observations, body weight and body weight change, clinical signs determinations, gross necropsy, viral shedding on oral swabs by RT-qPCR, viral burden in lung tissue by TCID₅₀ virus titer, and viral genome copy number in lungs by RT-qPCR and lung histopathology analysis.

No mortality was SARS-CoV-2 disease-associated.

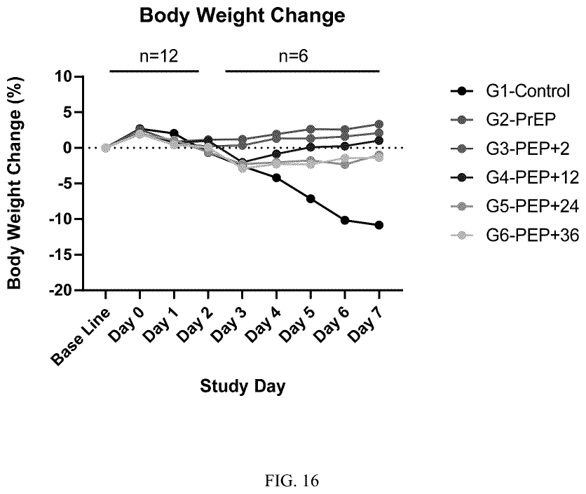

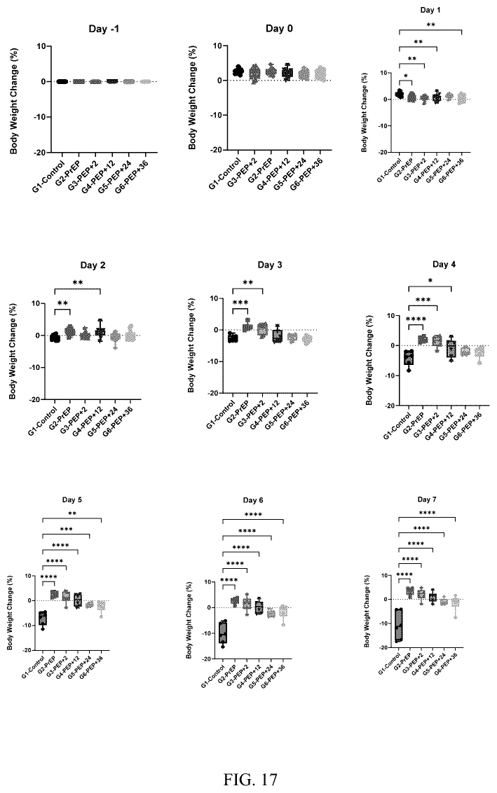

Animals in the control Group 1 designated as vehicle presented an average of −10.84% body weight loss, while all the treatment groups had a max of −1.39% BWL which was seen in Group 6. Groups 2 (+3.31% ), 3 (+2.09% ) and 4 (+1.02% ) all gained weight over the course of the study (FIG. 16). Day −2 and Day −1 showed no differences between any of the groups. On Day 1, Groups 2 (P≤0.0272, *) 3 (P≤0.0038, **), 4 (P≤0.0076, **), and 6 (P≤0.0015, **) all presented significant body weight loss compared to the vehicle control Group 1 (FIG. 17). From Day 2 to Day 4 significant differences (P≤0.0029, **; P≤0.0003, ***; P≤0.0001****) were shown consistently in Group 2, which was dosed as a intranasal prophylactic antiviral with 5 mg/kg of DCOY101 starting 48 hours prior to challenge through day 6. During Study Day 5 to Day 7 strong significant differences were observed that were consistent with a canonical time-response curve in the postexposure groups (Groups 3 to 6). Briefly, Group 2 (pre-treatment −48 hrs) had an average body weight change of +3.31% while Group 6 (post-treatment+36 hours) had an average body weight change of −1.39%.

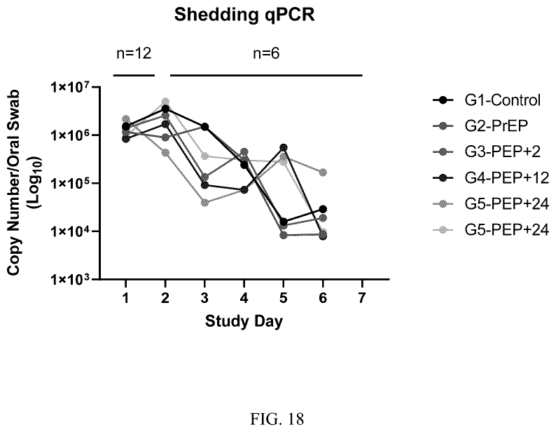

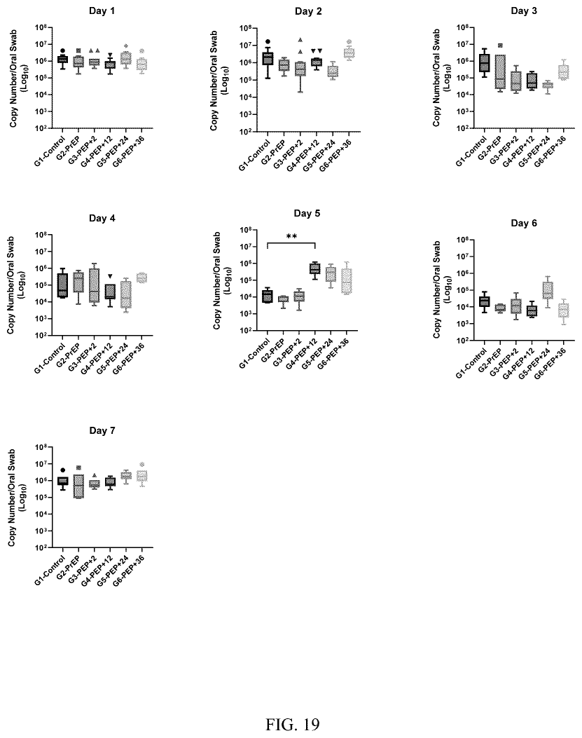

Viral shedding in oral swabs was estimated as viral genome copy number analyzed by RT-qPCR from Day 1 to Day 6. Data from Day 7 were excluded from analysis due to lack of reliability. Summary of the shedding profiles are shown in FIG. 18. There are some numerical differences with respect to the Control Group 1, especially on Day 3, but no statistical differences could be found in any of the groups on Days 1, 2, 3, 4, or 6 except on Day 5, where Group 4 (Pre-exposure+36 hrs) showed (** P≤0.0030) significant increase when compared to the control, Group 1. The detailed information about the Oral Swabs by RT-qPCR per day is shown in FIG. 19.

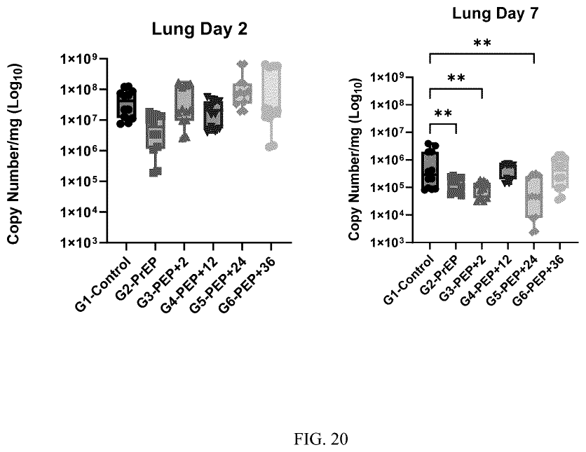

Briefly, RT-qPCR (using primers in Table 4 above) titers from lung samples showed some numerical differences, especially in the pre-exposure treatment (PrEP) but again no significant differences. However, on Day 7, there is a decrease with significant differences (P≤0.0028, **; P≤0.0017, ** and P≤0.0019, **) in Groups 2 (PrEP), 3 (PEP+2) and 5 (PEP +24) in the lung tissue collected compared to the vehicle control Group 1 as shown in FIG. 20.

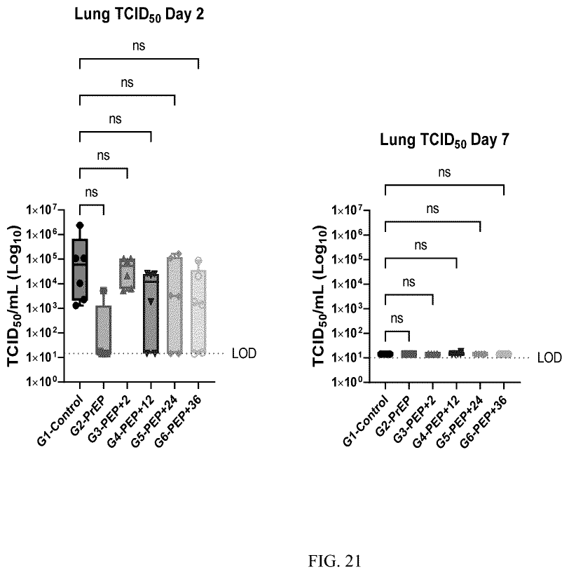

Viral load in lungs were estimated by TCID₅₀ assay on Days 2 and 7. On Day 2 there was some numerical differences, most of the animals (n=5/6) in group 2 (PrEP) show below detection limit titers, but one animal had an abnormal distance from the other values producing a global no significant reduction in the viral load accumulation in lungs when compared to the Group 1 (Vehicle Control) (FIG. 21). In Groups 4, 5 and 6 same phenomenon was observed 2 out of 6 animals presented lower titers, consistent with antiviral efficacy, but not all the animals follow the same consistency. These results are consistent to the RT-qPCR readouts. On Day 7, infectious virions were not detected by the TCID₅₀ assay in any of the groups FIG. 21; however, remaining RNA viral genomes or pieces are being still being detected by RT-qPCR in a lower quantity FIG. 20.

Based on the remarkable reduction of body weight loss, the data in this study suggest that intranasal administration once a day to hamsters for 6 days of 50 μl of DCOY101 at 5 mg/kg pre-exposure or post exposure up to 36 hours after challenge conferred partial protection against the B.1.617.2 variant of SARS-CoV-2 as shown by reduction in viral burden in lungs by TCID₅₀ on Day 2 and viral genome copy number in lung at Day 7 and in lower degree by lower virus copy number in oral swabs on Day 3, when compared to the vehicle control group G7. DCOY101 appears to reduce SARS-CoV-2-disease pathogenesis but does not seem to have an impact on viral load in lungs or reduce viral shedding in the hamster model under the conditions tested.

### Example 4-Efficacy of Potential Antiviral Compounds Against Betacoronavirus In Vitro

The objective of this study is to test the in vitro efficacy of DCOY101 as potential antiviral therapeutic against β-CoVs: SARS-CoV-1, MERS-CoV, WA1, Delta and Omicron variants of SARS-CoV-2.

The test articles (TA) are identified in the Table 7.

TABLE 7
Test Article Identification

| No | ID of TA | Molecular Weight | Solubility |
|---|---|---|---|
| 1 | DCOY101 | 10336.6 g/mol | DMSO |
| 2 | EK1 | 10983.6 g/mol | DMSO |

Test articles were provided in lyophilized powder and diluted at 10 mg/ml (or 967.49 μM) stock concentration in DMSO per the scheme in Table 8 and, following dilution, will be in 1× media (1× DMEM with 2% FBS.

TABLE 8
Test Article Dilution Scheme

| | | 2x initial TA | 1x initial TA |
|---|---|---|---|
| No. | Dilution | conc. (μM) | conc. (μM) |
| 1 | Neat | 200 | 100 |
| 2 | 1:3 | 66.6 | 33.3 |
| 3 | 1:9 | 22.2 | 11.1 |
| 4 | 1:27 | 7.4 | 3.7 |
| 5 | 1:81 | 2.4 | 1.2 |
| 6 | 1:243 | 0.82 | 0.41 |
| 7 | 1:729 | 0.27 | 0.13 |
| 8 | 1:2187 | 0.09 | 0.04 |

The dose-dependent antiviral effect was studied at a target MOI of 0.005 TCID₅₀/cell. Viruses were grown and tittered in African green monkey kidney (Vero E6) cells, Human Epithelial Cells (Calu-3), human fibroblasts (MRC-5), Macaca mulatta monkey rhesus kidney cells (LLC-MVK2), or colorectal epithelial adenocarcinoma human cells (Caco-2) as listed in Table 9. Cells were maintained in Dulbecco's Minimum Essential Medium with 10% Fetal Bovine Serum and antibiotics. For the efficacy assay the FBS was reduced to 2%.

TABLE 9
Viruses

| | | | BEI | Cell | TCID₅₀ | | |
|---|---|---|---|---|---|---|---|
| No. | ID. | Strain/Isolate | No. | Line | c.d.# | Detection | BSL |
| 1 | WA1 | hCoV-19/USA- | NR- | Vero | 200 | Antibody | 3 |
| | | WA1/2020 | 52281 | E6 | | | |
| | | (Lineage A.1) | | | | | |
| 2 | Delta | USA/MD- | NR- | Vero | 200 | Antibody | 3 |
| | | HP05647/2021 | 55672 | E6 | | | |
| | | (Lineage | | | | | |
| | | B.1.617.2) | | | | | |
| 3 | Omnicron | hCoV- | NR- | Calu-3 | 400 | Antibody | 3 |
| | | 19/USA/MD- | 56461 | | | | |
| | | HP20874/2021 | | | | | |
| | | (Lineage | | | | | |
| | | B.1.1.529) | | | | | |
| 4 | P1 | hCoV- | NR- | Vero | 200 | Antibody | 3 |
| | | 19/Japan/TY7- | 54982 | E6 | | | |
| | | 503/2021 (P.1.) | | | | | |
| 5 | SARS- | SARS-1 Strain: | NR- | Vero | 200 | Antibody | 3 + |
| | CoV-1 | Urbani 200300592 | 18925 | E6 | | | BSAT |
| 6 | MERS-CoV | EMC/2012 | NR- | Vero | 200 | Antibody | 3 |
| | | | 44260 | E6 | | | |
| 7 | OC43 | OC43 | NR- | VAT | | Antibody | 2 |
| | | | 52725 | | | | |
| 8 | 229E | 229E | NR- | MRC-5 | | Antibody | 2 |
| | | | 52726 | | | | |
| 9 | NL63 | NL3 or | NR-470 | LLC-MK2 | | Antibody | 2 |
| | | Amsterdam 1 | | or Caco2 | | | |

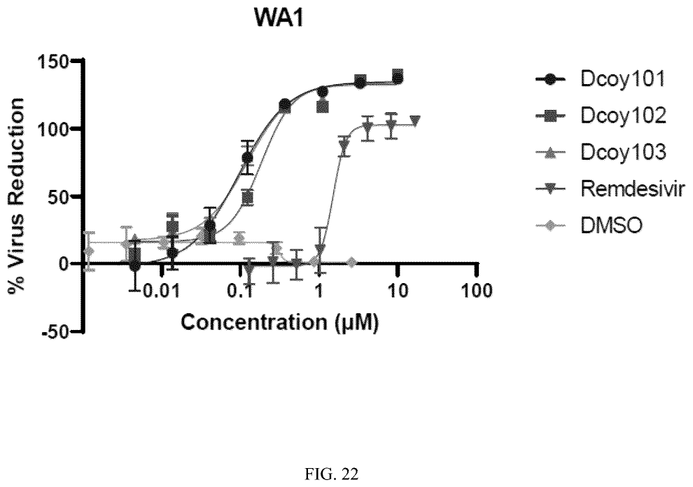

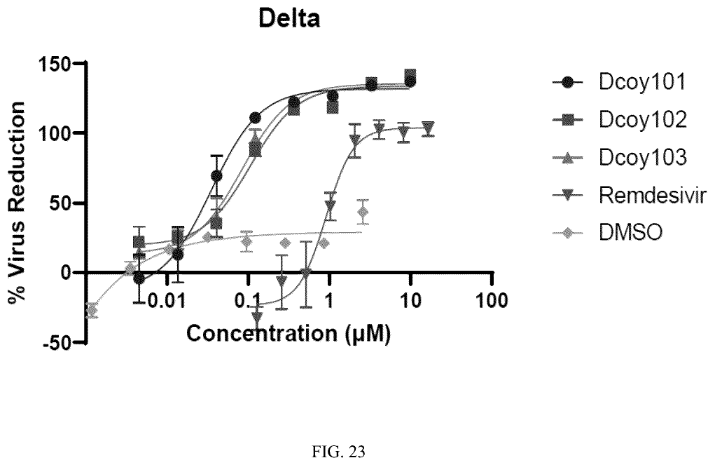

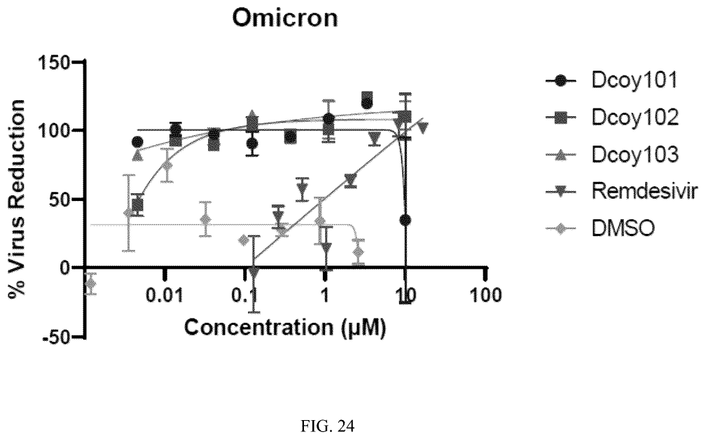

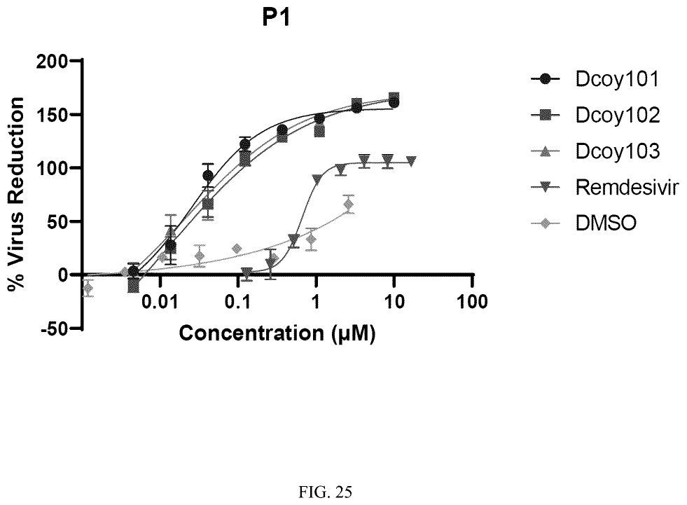

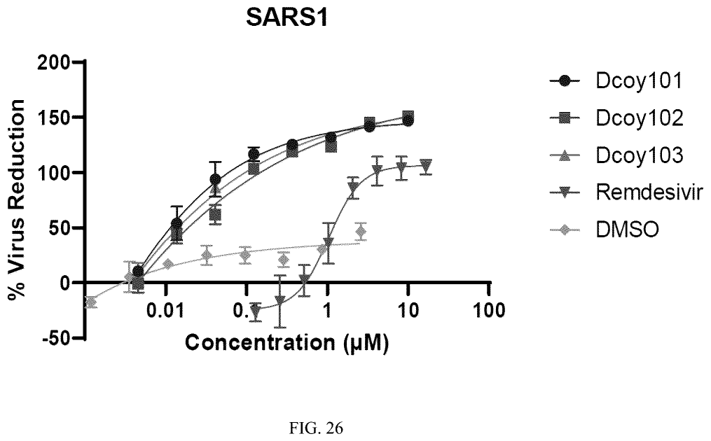

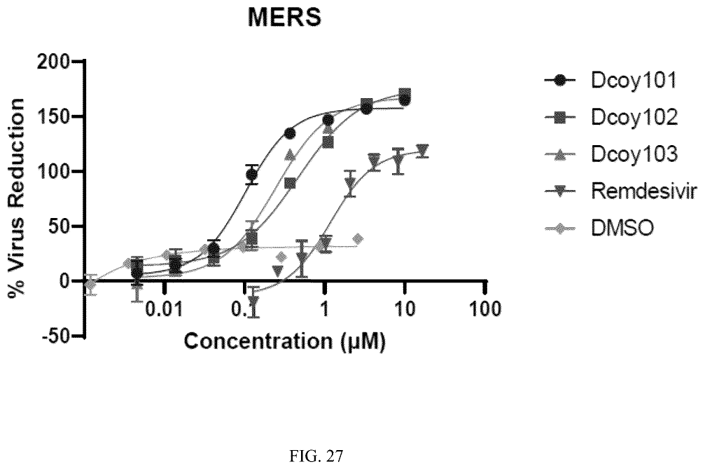

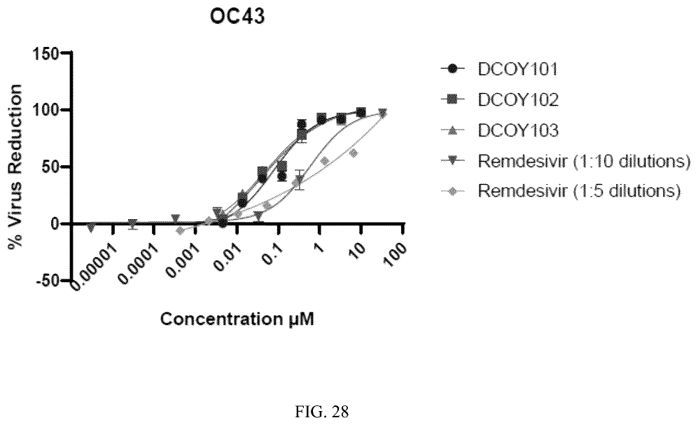

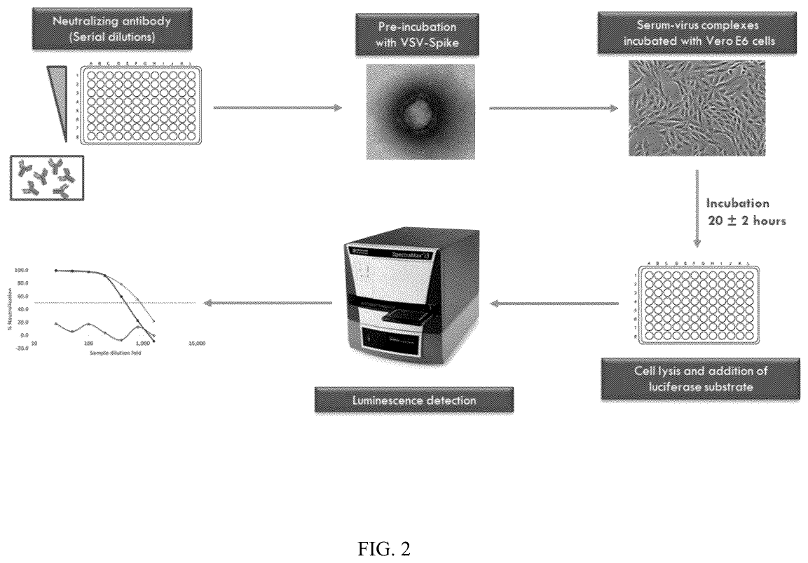

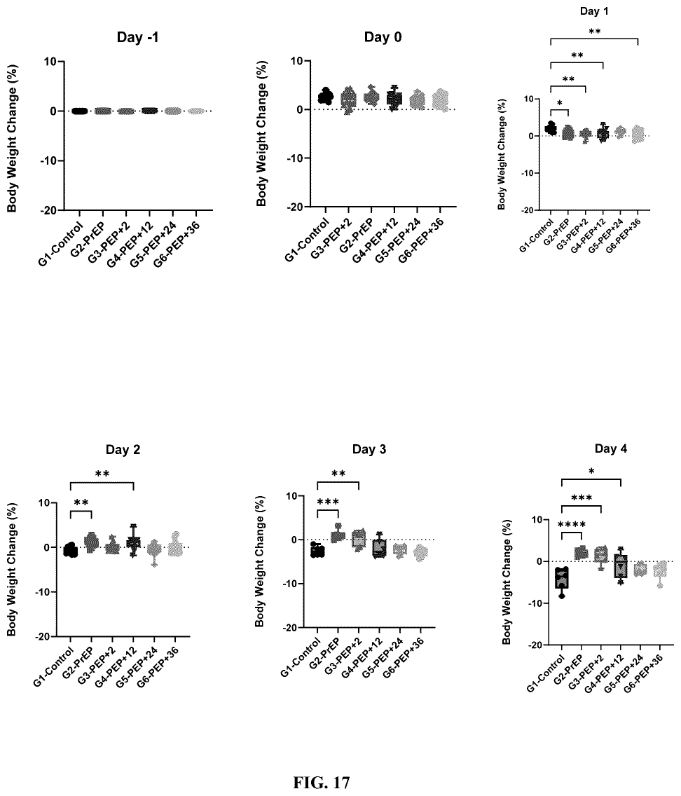

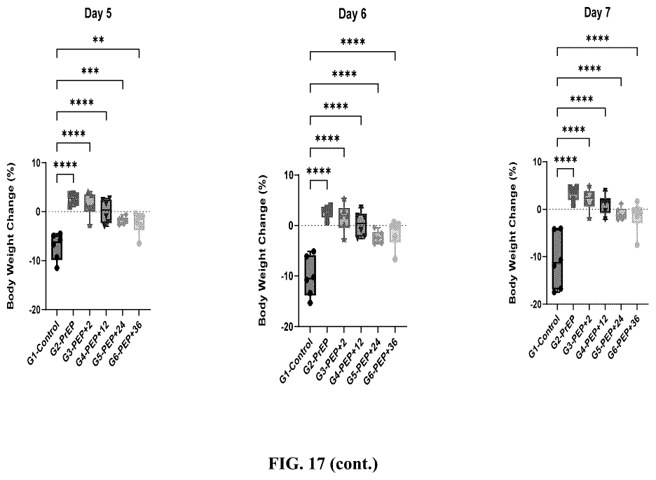

Antiviral activity was determined for each test article using a pre-post treatment regimen. TA was mixed with virus and incubated for 60±10 min before being added to a monolayer of confluent Vero E6 cells in triplicate. TA:virus was then added to the cell monolayer to allow for viral adsorption for 60-90 min. Following the adsorption, cells were washed with 1× PBS or media and 1× media with TA was replaced on top of the cells. Remdesivir free-base was evaluated in parallel as a positive control compound. Cells with DMSO were evaluated in parallel as a negative control. Results are shown in FIGS. 22-28.

While this invention has been particularly shown and described with references to preferred embodiments thereof, it will be understood by those skilled in the art that various changes in form and details may be made therein without departing from the scope of the invention encompassed by the appended claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference. The relevant teachings of all patents, published applications and references cited herein are incorporated by reference in their entirety.

1. A compound having the formula:
(Peptide-Linker)ₙ-B-Hydrophobic Moiety
wherein each Peptide is independently a HRC Peptide or a targeting peptide, provided that at least one peptide is a HRC Peptide, each Linker is independently a bivalent linking moiety, B is a multivalent moiety comprising cysteine, X, and optionally Y, and/or optionally Z, wherein X, Y and Z are defined herein, and n is an integer selected from 1, 2, 3 or more.

2. The compound of claim 1, wherein the compound comprises a targeting peptide and one or more HRC Peptide.

3. The compound of claim 1, wherein the targeting peptide is an ACE2 targeting peptide or a receptor binding domain peptide.

4. The compound of claim 1, wherein the Hydrophobic moiety is cholesterol.

5. The compound of claim 1, wherein the Z does not comprise a structure according to formula (I):

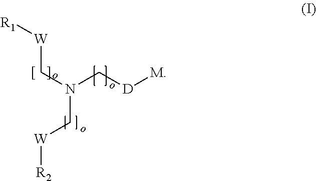

6. The compound of claim 1, wherein the compound has the following structure 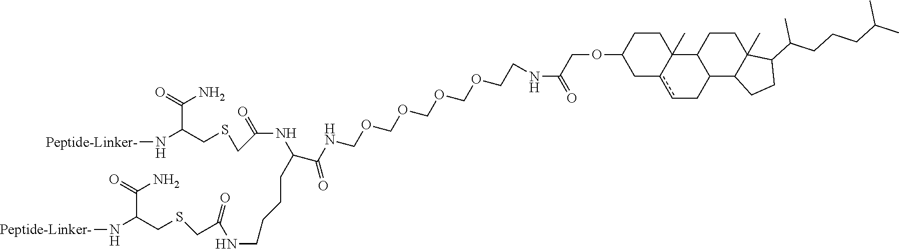
wherein the peptide-linker is DISGINASVVNIQKEIDRLNEVAKNLNESLIDLQEL-GSGSG-.

7. (canceled)

8. A pharmaceutical composition comprising a compound of claim 1 and a pharmaceutically acceptable carrier.

9. A method treating respiratory infection associated with a SAR-Cov-2 variant in a subject in need thereof, the method comprising administering to the subject an effective amount of a compound having the formula:
(Peptide-Linker)ₙ-B-Hydrophobic Moiety
wherein each Peptide is independently a HRC Peptide or a targeting peptide, provided that at least one peptide is a HRC Peptide, each Linker is independently a bivalent linking moiety, B is a multivalent moiety comprising cysteine, and X, and optionally Y, and/or optionally Z, wherein X, Y and Z are defined herein, and n is an integer selected from 1, 2, 3 or more; and
wherein the variant comprises at least 5 mutations wherein the at least 5 mutations are independently in the spike protein S1 subunit or the S2 subunit or combinations thereof.

10. The method of claim 9, wherein the compound comprises a targeting peptide and one or more HRC Peptide.

11. The method of claim 9, wherein the targeting peptide is an ACE2 targeting peptide or a receptor binding domain peptide.

12. The method of claim 9, wherein the Hydrophobic Moiety is cholesterol.

13. The method of claim 9, wherein the at least 5 mutations are independently in N-Terminal domain (NTD), the receptor binding domain (RBD), the fusion peptide (FP)domain, the heptad repeat 1 (HR1) domain, or combinations thereof.

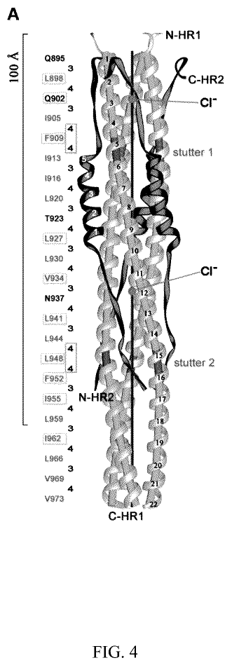

14. The method of claim 9, wherein the at least 5 mutations are independently selected from the mutations listed in FIG. 4.

15. The method of claim 14, wherein the at least 5 mutations are independently selected from the group consisting of:
(i) at least 5 mutations from the SAR-Cov-2 Alpha variant;
(ii) at least 5 mutations from the SAR-Cov-2 Beta variant;
(iii) at least 5 mutations from the SAR-Cov-2 Delta variant; and
(iv) at least 5 mutations from the SAR-Cov-2 Omicron variant.

16. The method of claim 13, wherein the variant comprises at least 10 mutations.

17. The method of claim 13, wherein the variant comprises at least 15 mutations.

18. The method of claim 13, wherein the variant comprises at least 20 mutations.

19. The method of claim 9, wherein the SARS-CoV-2 comprises at least one variant selected from B.1.1.7 (Alpha), B.1.351 (Beta), P.1 (Gamma), B.1.617.2 (Delta), B.1.429/B.1.427 (Epsilon), B.1.617.1 (Kappa), B.1.525 (Eta), B.1.526 (Iota), P.3 (Theta), P.2 (Zeta), and B.1.1.529 (Omicron).

20. The method of claim 19, wherein the SARS-CoV-2 comprises at least one variant selected from A.1-A.6, B.3-B.7, B.9, B.10, B.13-B.16, B.2, B.1 lineage, P.1, P.2, P.3, and R.1.

21. The method of claim 20, wherein the B.1 lineage comprises at least one of (including, but not limited to, B.1, B.1.1, B.1.1.7, B.1.1.7 with E484K, B.1.2, B.1.5-B.1.72, B.1.9, B.1.13, B.1.22, B.1.26, B.1.37, B.1.3-B.1.66, B.1.177, B.1.243, B.1.313, B.1.351, B.1.427, B.1.429, B.1.525, B.1.526, B.1.526.1, B.1.526.2, B.1.617, B.1.617.1, B.1.617.2, B.1.617.3, B.1.619, B.1.620, and B.1.621.

22. The method of claim 9, wherein the administration is achieved using an intranasal spray, an inhaler or a nebulizer.

23. The method of claim 9, wherein the compound is administered in combination with at least one other antiviral active agent or therapy.

24. The method of claim 9, wherein the compound has the following structure 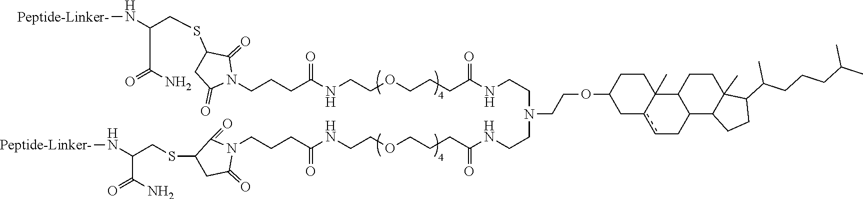
wherein the peptide-linker is DISGINASVVNIQKEIDRLNEVAKNLNESLIDLQEL-GSGSG-.

25. (canceled)

26. (canceled)
